(19) United States
(12) Patent Application Publication
(10) Pub. No.: US 2010/0310451 A1
(43) Pub. Date: Dec. 9, 2010

(54) ALTERED N-CADHERIN PROCESSING IN TUMOR CELLS BY FURIN AND PROPROTEIN CONVERTASE 5A (PC5A)

(75) Inventors: Deborah Maret, Montreal (CA); David R. Colman, Westmount (CA); Eugenia Gruzglin, New York, NY (US); Nabil Seidah, Verdun (CA)

Correspondence Address: OGILVY RENAULT LLP, 1, Place Ville Marie, SUITE 2500, MONTREAL, QC, H3B 1R1, CA

(73) Assignees: THE ROYAL INSTITUTION FOR THE ADVANCEMENT OF LEARN, Montreal, QC (CA); IRCM (INSTITUT DE RECHERCHES CLINQUES DE MONTRÉAL), Montreal, QC (CA); MOUNT SINAI SCHOOL OF MEDICINE, New York, NY (US)

(21) Appl. No.: 12/740,654

(22) PCT Filed: Nov. 3, 2008
(86) PCT No.: PCT/CA2008/001949

§ 371 Date: Aug. 26, 2010

Related U.S. Application Data
(60) Provisional application No. 60/996,119, filed on Nov. 1, 2007.

(51) Int. Cl. A61K 51/00; G01N 33/574; C12Q 1/68; A61K 49/00; A61K 49/06

(52) U.S. Cl. 424/1.11; 435/7.23; 435/6; 424/9.1; 424/9.3

(57) ABSTRACT

The present invention relates to a method for diagnosis and/or prognosis of cancer and for monitoring the progression of cancer and/or the therapeutic efficacy of an anti-cancer treatment in a subject by determining the molecular form of cadherin at the cell surface of cancer cells in the subject. The invention also relates to a method for preventing, inhibiting or treating cancer or its metastasis in a subject by increasing the adhesive forms of cadherin and/or decreasing the non-adhesive forms of cadherin at the cell surface. The invention also relates to a method step of determining the expression level of furin and proprotein convertase 5A (PC5A).

### TECHNICAL FIELD

The present invention relates to a method for diagnosis and prognosis of cancer and for monitoring the progression of cancer and/or the therapeutic efficacy of an anti-cancer treatment in a subject by detecting altered cadherin proteins in tumor cells. Therapeutic methods for preventing, inhibiting or treating cancer are also presented herein.

### BACKGROUND OF THE INVENTION

The transformation of a normal cell into a malignant cell results, among other things, in the uncontrolled proliferation of the progeny cells, which exhibit immature, undifferentiated morphology, exaggerated survival and proangiogenic properties and expression, and overexpression or constitutive activation of oncogenes not normally expressed in this form by normal, mature cells. Once a tumor has formed, cancer cells can leave the original tumor site and migrate to other parts of the body via the bloodstream or the lymphatic system or both by a process called metastasis. In this way the disease may spread from one organ or part to another non-contiguous organ or part.

The increased number of cancer cases reported around the world is a major concern. Currently there are only a handful of treatments available for specific types of cancer, and these provide no guarantee of success. In order to be most effective, these treatments require not only an early detection of the malignancy, but a reliable assessment of the severity of the malignancy, the ability of the malignancy to spread, and the response of a subject to anti-cancer treatment. There is a need for diagnostic and prognostic tools to identify and characterize tumors, and which can be used to assign treatments to a patient, and to monitor therapeutic efficacy. There is also a need for anti-cancer treatments which can be administered to subjects having cancer to prevent, inhibit or treat the disease and the spread thereof.

During the process of tumor progression, a subset of primary tumor cells undergoes molecular changes leading to an increased ability to survive, proliferate, invade, and in many tumors, form secondary metastases. The mechanisms governing invasion and metastasis are complex and poorly understood. However, it is recognized that at the cell surface, alterations in classes of adhesion molecules are critical for detachment of tumor cells, mobility through host tissue, and the successful formation of secondary sites (Christofori (2006) Nature 441: 444-450). These alterations involve not only reduction in surface adhesion molecules, but also changes in the profile of adhesion molecule expression at the cell surface.

Classical cadherins are cell adhesion molecules (CAMs) that mediate Ca2+-dependent, and generally, homophilic intercellular interactions. They have been identified as key CAMs in epithelia, since they are critical for establishing and maintaining intercellular connections and for the spatial segregation of cell types. The precursor form of classical cadherins contains a signal sequence that is cleaved in the rough endoplasmic reticulum to reveal a prodomain of 130 amino acids (Koch et al. (2004) Structure 12: 793-805). Proteolytic processing of the prodomain is necessary to generate adhesively competent cadherins at the cell surface (Ozawa and Kemler (1990) J Cell Biol 111: 1645-1650). The recently solved N-cadherin prodomain structure (Koch et al. (2004) Structure 12: 793-805) reveals that the prodomain lacks the essential structural features for cadherin adhesion, thus explaining why it cannot itself mediate adhesive interactions, and why its presence prior to cleavage proximal to the mature cadherin sequence protects from dimerization of cadherins intracellularly ((Ozawa and Kemler (1990) J Cell Biol 111: 1645-1650); Wahl et al (2003) J Biol Chem 278: 17269-17276).

Classical cadherins play important roles in the pathogenesis of cancer, and it has been shown that the metastatic potential of tumor cells inversely correlates with expression of cadherins. In the skin, E-cadherin normally mediates attachment of melanocytes to keratinocytes, and is critical for intercellular signaling between these two cell types (Hsu et al. (2000) Am J Pathol 156: 1515-1525). In melanoma, malignant vertical growth phase (VGP) cells lose E-cadherin expression, whereas N-cadherin levels significantly increase and persist throughout malignant transformation. It has been concluded that E-cadherin downregulation in VGP melanoma cells, along with the upregulation of adhesively competent N-cadherin, enables invasion into the dermis and the subsequent formation of secondary metastases by a subset of these cells.

An E- to N-cadherin switch also takes place in other types of carcinomas. Loss of E-cadherin has been shown to be associated with high tumor grades and poor prognosis, and the upregulation of N-cadherin correlates with induced cellular motility. In addition to the upregulation of N-cadherin following loss of E-cadherin, the emergence of cadherin-11 in malignant carcinomas such as breast and prostate, correlates with invasiveness and poor prognosis. N-cadherin and cadherin-11 have been referred to as “mesenchymal cadherins” to denote the invasive morphology of cells bearing these cadherins on their surfaces, compared to polarized epithelial cells (Thiery (2002) Nat Rev Cancer 2: 442-454).

It is believed therefore that loss of E-cadherin and the upregulation of mesenchymal cadherins promote tumor cell invasion and metastasis. It has been hypothesized that loss of E-cadherin may be a prerequisite for tumor cell invasion, since E-cadherin functions in “anchoring” normal cells in place (Birchmeier and Behrens (1994) Biochem Biophys Acta 1198: 11-26). Re -establishing adherens junctions by forced E-cadherin expression, results in a reversion from an invasive, mesenchymal, to a benign, epithelial phenotype. Thus, loss of E-cadherin results in the disruption of adhesion junctions between adjacent cells allowing malignant cells to detach from the “E-cadherin” epithelial cell layer and invade the host tissue.

The gain of expression of mesenchymal cadherins such as N-cadherin, is thought to mediate adhesion of malignant cells to N-cadherin expressing stromal or endothelial cells, rather than epithelial cells, facilitating invasion of tumor cells and the formation of secondary metastases (Qi et al. (2005) Molec Biol Cell 16: 4386-4397). It has also been proposed that the association of tumor cells with fibroblasts and endothelial cells induces these host cells to produce growth factors and/or proteases promoting growth and invasion of the tumor cells (Li et al. (2002) Crit. Rev Oral Biol Med 13: 62-70). In breast cancer cells, it has been shown that the N-cadherin invasive activity is partially due to an interaction with the FGF receptor at the cell surface, resulting in sustained activation of the MAPK-ERK pathway as well as other pathways, and increased expression of MMP-9 (Suyama et al. (2002) Cancer Cell 2: 301-314).

Other tumors do not undergo an E- to N-cadherin shift, but exhibit persistence of N-cadherin in their component cells normally, as well as in the highly malignant state. A particularly interesting model is primary brain tumors, which arise from cells derived from the primitive neuroepithelium, and are among the most devastating malignancies. Glioblastoma multiforme (GBM) is the most aggressive type of malignant glioma, and long-term survival is seldom observed due to the extensive infiltration of vital brain regions by subpopulations of highly invasive cells. These tumors invade throughout the brain tissue as single cells, with a predilection for migration along existing anatomical structures, such as white matter tracts, the subpial glial space, and the periphery of neurons and blood vessels, and almost never metastasize outside the brain. Dissemination of glioma cells within the brain appears to depend on complex interactions, and possibly cooperation with resident brain cells, and likely correlates with CAM profiles. An upregulation of N-cadherin in malignant glioma cells compared to normal brain tissue has been demonstrated (Asano et al. (2004) J Neuro-Oncol 70: 3-15).

It is known that destabilized cell contacts, cellular reorganization, and metastatic dissemination are all associated with changes in cell adhesion, and that cadherins such as N-cadherin are major cellular adhesion molecules (CAMs) in normal physiology and during tumorigenesis, and have been shown to possess a range of adhesive strengths. However, this hierarchy of adhesion has been believed to be regulated solely by monomer: dimer ratios (Tanaka et al. (2000) Neuron 25: 93-107), “overlapping” domains (Sivasankar et al. (1999) Proc Natl Acad Sci 96: 11820-11824), clustering (He et al. (2003) Science 302:109-113), and by mass amounts of cadherin on cell surfaces. Persistence of the prosequence has never been observed, and it has been believed that the N-terminal prosequence in classical cadherins is completely removed by an endoprotease within the late Golgi following association of the catenins, resulting in a mature, adhesively competent molecule at the cell surface.

However, the idea that upregulation of adhesively competent N-cadherin mediates invasion is not easily reconciled with data showing that increased N-cadherin levels are associated with stronger intercellular adhesion and decreased cell motility (Gumbiner (1996) Cell 84: 345-357). There is a need therefore to understand better the role of N-cadherin in the invasion and migration of tumor cells, and in the stages of malignancy which occur during tumor progression.

It would also be highly desirable to be provided with a diagnostic method and/or a prognostic tool that permits evaluation of the invasiveness of a tumor and of the stage of malignancy of the tumor.

### SUMMARY OF THE INVENTION

The present invention relates to a method for diagnosis and prognosis of cancer and for monitoring the progression of cancer and/or the therapeutic efficacy of an anti-cancer treatment in a subject by detecting altered cadherin proteins in tumor cells, as well as therapeutic methods for preventing, inhibiting or treating cancer.

In accordance with the present invention, there is provided a method for diagnosing or determining prognosis of a cancer in a subject, comprising determining the molecular form of cadherin at the cell surface of cancer cells in the subject, wherein the presence of a non-adhesive form of cadherin indicates that the cancer is invasive or metastatic.

Also in accordance with the present invention, there is provided a method for diagnosing or determining prognosis of a cancer in a subject, comprising determining the molecular form of cadherin at the cell surface of cancer cells in the subject, wherein a high ratio of non-adhesive to adhesive forms of cadherin indicates that the cancer is invasive or metastatic.

Further in accordance with the present invention, there is provided a method for diagnosing or determining prognosis of a cancer in a subject, comprising determining the expression level of furin and/or PC5 in cancer cells in the subject, wherein low expression of furin and/or high expression of PC5 indicates that the cancer is invasive or metastatic. There is also provided a method for monitoring the progression of a cancer in a subject, the method comprising determining the molecular form of cadherin at the cell surface of cancer cells in the subject, wherein the presence of a non-adhesive form of cadherin or a high ratio of non-adhesive to adhesive forms of cadherin indicates that the cancer has progressed to a metastatic phase.

In another aspect, there is provided herein a method for monitoring the efficacy of an anti-cancer treatment in a subject, comprising determining the molecular form of cadherin at the cell surface of cancer cells in the subject at a first timepoint, determining the molecular form of cadherin at the cell surface of cancer cells in the subject at a second timepoint, and comparing the amounts of non-adhesive and adhesive cadherin at the first and second timepoints, wherein a decrease or no change in the amount of non-adhesive cadherin or an increase in the amount of adhesive cadherin in the second sample compared to the first sample indicates efficacy of the anti-cancer treatment.

In yet another aspect, there is provided a method for monitoring the efficacy of an anti-cancer treatment in a subject, comprising determining the expression level of furin and/or PC5 cancer cells in the subject at a first timepoint, determining the expression level of furin and/or PC5 cancer cells in the subject at a second timepoint, and comparing the expression levels of furin and/or PC5 at the first and second timepoints, wherein an increase in the expression levels of furin and/or a decrease in the expression levels of PC5 in the second sample compared to the first sample indicates efficacy of the anti-cancer treatment.

In an embodiment, the encompassed cancer is selected from the group consisting of melanoma, breast cancer, prostate cancer, bladder cancer, squamous cell cancer, and malignant glioma.

In another embodiment, the encompassed cadherin is a type I or type II classical cadherin. The cadherin may be selected from the group consisting of E-cadherin, N-cadherin, R-cadherin, C-cadherin, VE-cadherin, P-cadherin, K-cadherin, T1-cadherin, T2-cadherin, OB-cadherin, Br-cadherin, M-cadherin, cadherin-12, cadherin-14, cadherin-7, F-cadherin, cadherin-8, cadherin-19, EP-cadherin (X1), BS-cadherin (Bs) and PB-cadherin (Rn). In a particular aspect, the cadherin is N-cadherin.

In another aspect, the molecular form of cadherin at the cell surface of cancer cells in the subject is determined in the methods of the invention using immunocytochemistry or immunoblotting in a sample from a subject. In a further aspect, the molecular form of cadherin at the cell surface of cancer cells in the subject is determined using radionuclide imaging, SPECT imaging, magnetic resonance imaging, fluorescence imaging, positron emission tomography, CT imaging, or a combination thereof.

In accordance with the present invention, there is also provided a kit for diagnosing or determining prognosis of a cancer in a subject, comprising reagents for determining the molecular form of cadherin at the cell surface of cancer cells in the subject, and instructions for use thereof. The kit may contain reagents comprising an antibody specific for a non-adhesive cleavage form, e.g., an antibody specific for the pro-domain of a cadherin, e.g. the anti-proN antibody. In another embodiment, the kit may contain reagents for determining expression levels of furin or PC5 in cancer cells in the subject, and instructions for use thereof. For example, the kit may contain reagents comprising PCR reagents, primers, antibodies specific for furin or PC5, and/or reagents for assaying furin or PC5 enzymatic activity.

In accordance with the present invention, there is also provided a method for preventing, inhibiting, or treating cancer or its metastasis comprising administering to a subject in need thereof an effective amount of an agent, wherein the agent increases the amount of adhesive cadherin or decreases the amount of non-adhesive cadherin at the cell surface of cancer cells in the subject. In one aspect, the agent may be an inhibitor of PC5 or an activator of furin. In another aspect, the agent is furin. In yet another aspect, the agent is an antisense against PC5 RNA, siRNA against PC5, or a small molecule inhibitor of PC5.

Also provided herein is the use of an agent which increases production of adhesive cadherin forms or decreases production of non-adhesive cadherin forms for preventing, inhibiting or treating cancer or metastasis thereof. The invention also relates to the use of an agent which increases production of adhesive cadherin forms or decreases production of non-adhesive cadherin forms in the manufacture of a medicament for preventing, inhibiting or treating cancer or metastasis thereof. In another aspect, a pharmaceutical composition comprising an agent which increases production of adhesive cadherin forms or decreases production of non-adhesive cadherin forms, and a pharmaceutically acceptable carrier is provided herein.

### BRIEF DESCRIPTION OF THE DRAWINGS

Having thus generally described the nature of the invention, reference will now be made to the accompanying drawings, showing by way of illustration, an embodiment or embodiments thereof, and in which:

FIG. 1 illustrates the expression of precursor N-cadherin on the surface of highly invasive glioma and metastatic melanoma cells, wherein: in (A) and (B) it is shown Western blots examining N-cadherin levels in U343 and U251 glioma cells, and in WM115 VGP melanoma, and WM266 metastatic melanoma cells, using an Ncad cytoplasmic Ab; results were quantified by densitometric analysis and demonstrate that comparable levels of N-cadherin are expressed in more invasive and less invasive glioma cells, and during melanoma malignant progression; in (C) and (D) is shown an aggregation assay of glioma and melanoma cells, in the presence of calcium, alone or in combination with L-cells overexpressing N-cadherin (LN cells); in mixing experiments, tumor cells were labelled with Dil and LN cells were labelled with DiO; results were quantified as percent of single cells relative to t=0 min, and demonstrate that U343 cells and WM115 cells exhibit high aggregation relative to U251 and WM266 cells, respectively, and the rate of aggregation increased in mixing experiments, especially in U251 and WM266 cells; values are means ±SEM; in (E) is shown immunocytochemistry demonstrating intracellular localization of proN in permeabilized tumor cells (top panels), and substantial surface localization only in U251 and WM266 non-permeabilized (bottom panels), live tumor cells, Bar, 10 μm; in (F) is shown Western blot analysis of surface biotinylated U343 and 0251 cells; Ncad cytoplasmic Ab detected total Ncad protein, proN Ab detected precursor protein, and Erk (p42/44) Ab was a marker for cytoplasmic proteins; experiments were carried out with 90 or 60 pg of total protein and the proportion of surface to total proN was determined, and found to be approximately 80% in U251 cells and only 25% in U343 cells; values are means ±SEM.

FIG. 2 illustrates the cell surface precursor N-cadherin promotes migration and invasion of tumor cells, wherein: in (A) is shown a schematic diagram of precursor N-cadherin protein with endogenous furin recognition site, and the engineered factor Xa cleavage site; Tryptophan at position 2 is in bold; in (B) is shown immunocytochemistry demonstrating co-localization of mutant Ncad-I in stably transfected WM115, WM266 and U343 cells, with GFP or with c-myc in melanoma and glioma, respectively; Bar, 10 μm; in (C) is shown a wound healing assay which was carried out with proN (a rabbit polyclonal antibody specific for the N-cadherin prodomain) and mock transfected WM115 and WM266 cells; migrated cells at 6h with or without Factor Xa treatment were counted and the results were plotted relative to WM115-proN values; values are means ±SEM; in (D) is shown spheroids of proN and mock transfected U343 cells which were implanted into a collagen matrix; Invasion was monitered and quantified using concentric grids spaced by 150 μm, where the edge of the spheroid was designated as 0 μm; results obtained on day 3 post-implantation were plotted and demonstrate an increase in the number of surface proN expressing cells invading the collagen gel and abolishment of this effect upon treatment with factor Xa; values are means ±SEM; and in (E) is shown an aggregation assay of mutant proN transfected U343 cells or mock transfected cells, with or without Factor Xa treatment; results were quantified as percent of single cells relative to t=0 min, and demonstrate a substantial decrease in aggregate formation by cells expressing surface proN; values are means ±SEM.

FIG. 3 illustrates that Furin and PC5 proprotein convertases mediate cleavage of N-cadherin at the consensus site, and at a second site, respectively, wherein: in (A) is shown semiquantitative RT-PCR which was carried out to look at expression of PCs, and results show differential expression of furin and PC5A in U343 and U251 cells; GAPDH expression was used as a normalizing control; in (B) is shown real-time PCR which was carried out to quantify furin and PC5A expression; results are plotted as number of mRNA messages/106 S14 messages, and show contrasting furin and PC5A expression patterns in U343 and U251 cells; in (C) is shown HeLa cells which were transiently transfected with N-cadherin+/−FL-PC5A, or empty vector, and cells were incubated in the absence or presence of 50 μM dec-cmk; the conditioned medium was concentrated (20×) and run on a 15% gel and N-cadherin cleavage peptides were detected with the proN antibody; cleavage products with Mr's of 17 kDa and 20 kDa corresponded to processing of N-cadherin at the consensus site, and at the second site, respectively; as a positive control for PC inhibition by dec-cmk (Siegfried et al., 2003), HeLa cells were transfected with propDGF-A and incubated in the absence or presence of 50 μM dec-cmk (right panel); in (D) are shown transfections as in (C) which were carried out except that PC5A-ΔCRD was used instead of FL-PC5A; in (E-H) are shown transient transfections in HeLa cells which were carried out; PACE4 was used in (E), PACE4--ΔCRD was used in (F), furin in (G), and PC7 in (H); Cleavage at the second site was only mediated by PC5A with an intact CRD, and furin overexpression potentiated cleavage at the consensus site; in (I) it is shown that immunocytochemistry was carried out to look at PC5A localization in transfected HeLa cells; PC5A was in the pIRES2-EGFP vector, tagged with a V5 epitope, therefore cells were probed with anti-V5 antibody; cells were transfected with either FL-PC5A, or PC5A-ΔCRD; the green EGFP fluorescence is a control of PC5A transfection, whereas the red labeling indicates PC5A detection.

FIG. 4 illustrates that migration and aggregation of U251 and U343 cells depends on furin and PC5A expression, wherein: in (A) is shown U251 and U343 cells which were transfected with wt N-cadherin vector or empty vector and incubated in the presence or absence of the dec-cmk inhibitor; cleavage peptides resulting from N-cadherin processing were detected in the conditioned medium with the proN antibody, as in FIG. 3; N-cadherin was cleaved mostly at the second site in U251 cells, and exclusively at the first site in U343 cells; in (B) is shown immunocytochemistry of U251 and U343 cells that demonstrates localization of endogenous PC5A, and precursor-PC5A (pro-PC5A); an NT-PC5A antibody detected total PC5A protein, and an antibody against the PC5A prodomain detected only pro-PC5A; staining was carried out under non-permeabilizing conditions with or without heparin, or under permeabilizing conditions; in (C) is shown a wound healing assay that was carried out with mock transfected cells, or U251-furin cells, U343-PC5A cells, or U343-PC5A-R84A cells; in addition, this assay was carried out with U251 cells transfected with PC5A siRNA, and with U343 cells transfected with furin siRNA; migration was monitored over a 24h period, and results were quantified as number of migrated cells at 12h; in (D) it is shown that an adhesion assay was carried out with the stably transfected glioma cells, and siRNA transfected cells; cell aggregation was monitored over a 40 min time period and results were quantified as % single cells over time; values are means ±SEM.

FIG. 5 illustrates that the proprotein processing of N-cadherin by furin or PC5 determines the extent of cellular migration, wherein: in (A) is shown a schematic diagram of precursor N-cadherin protein with the endogenous second cleavage site, and the engineered mutant non-functional site (Ncad-II); Tryptophan at position 2, necessary for adhesion, is in bold; U343 cells were transiently transfected with wt N-cadherin, proN, or N-cadherin mutated at the second cleavage site (Ncad-II), with or without furin or PC5A convertase and wound healing in (B) and adhesion assays in (C) were carried out to determine the functional effects of N-cadherin processing by furin or PC5A at the consensus or the second cleavage site.

FIG. 6 illustrates that the carcinoma cell lines and aggressive primary brain tumor cells express cell surface precursor N-cadherin and variable levels of furin and PC5, wherein: in (A) is shown Western blot analysis of surface biotinylated primary brain tumor cells; OP128 and OP132 are highly aggressive glioblastoma multiforme (GBM), OP133 is a recurrent anaplastic oligodendroglioma, OP109 is a low grade glioma, and OP122 is an anaplastic astrocytoma; Ncad cytoplasmic Ab detected total Ncad protein, proN Ab detected precursor protein, and Erk (p42/44) Ab was a marker for cytoplasmic proteins; experiments were carried out with 60 μg of total protein and the proportion of surface to total proN was determined, and values are means ±SEM; in (B) is shown Western blot analysis of surface biotinylated metastatic prostate (PPC-1, PC3); bladder (JCA-1, T24), squamous cell (NC1-H226), and breast (MDA-MB-436) carcinomas; experiments were carried out and analyzed as in (A); in (C) it is shown that real-time quantitative PCR was carried out to look at furin and PC5A expression in a panel of primary brain tumors, and in carcinoma cells described in (B); CT-001 was a GBM, OP-132 was a GBM, OP-122 was an anaplastic grade III astrocytoma, OP-71 was a low grade glioma, CT-005 was a pilocytic astrocytoma, and OP-113 was a metastatic breast carcinoma; results are represented as the number of messages/106 S14 messages.

FIG. 7 illustrates a proposed schematic diagram depicting surface cadherin expression during melanoma and glioma progression, wherein: in I is shown that invasive radial growth phase (RGP) melanoma cells associate with each via E-cadherin mediated adhesion; An E- to N-cadherin switch gives rise to VGP melanoma which invade the dermis; cells with a high proportion of proN and inactivated N-cadherin are more invasive and have the ability to form secondary metastases; in II it is shown that glioma cells in the main tumor mass associate with each other via N-cadherin mediated adhesion; surface proN expression allows detachment and cells with a high proportion of the precursor protein are more invasive, but in general do not metastasize.

FIG. 8 illustrates that U251 human glioma cells exhibit extensive invasion compared to U343 glioma cells in a three-dimensional assay, wherein: spheroids of U343 and U251 glioma cells were implanted into a Type I collagen matrix, and invasion was measured on day 1 and day 5 post-implantation; Bar, 50 μm.

FIG. 9 illustrates aggregation of tumor cells alone, or mixed with L cells overexpressing N- or E-cadherin, wherein: in (A), aggregation assay of glioma and melanoma cells is shown, in the presence of calcium, demonstrating more extensive aggregation of U343 and WM115 cells, compared to U251 and WM266 cells; Bar, 50 μm; in (B) is shown an aggregation assay of tumor cells with LN cells or LE cells; tumor cells were labelled with Dil and L cells were labelled with DiO; results demonstrate co-aggregation of tumor cells with LN cells, and mutually exclusive segregation of tumor cells with LE cells; Bar, 50 μm.

FIG. 10 illustrates that mature and precursor N-cadherin protein exist on the same cell surface, wherein it is shown: immunocytochemistry demonstrating localization of N-cadherin, detected with NEC2 antibody (Ab), and localization of proN, detected with proN Ab, in permeabilized WM115 and WM266 melanoma cells.

FIG. 11 illustrates transient transfections of HeLa cells with N-cadherin and convertase enzymes, wherein the following is shown: Western blots of HeLa cells transfected with PC5A (A), with PC5AΔCRD (B), with PACE4 (C), with PACE4ΔCRD (D), with furin (E), or with PC7 (F); N-cadherin transfection was detected with anti-myc (9E10), and expression of either convertase was detected with anti-V5.

FIG. 12 illustrates stable transfections of glioma cells with convertase enzymes, wherein: in (A) U251 cells were stably transfected with furin, and U343 cells were stably transfected with PC5A or PC5A-R84A, and transfectants were selected for and expanded; transfected cells were detected by colocalization of either furin (labeled with anti-V5 Ab in red) and EGFP or PC5A (labeled anti-V5 Ab in red) and EGFP; in (B) U343 cells transfected with PC5A or PC5A-R84A were stained under non-permeabilizing conditions for surface localization of PC5A (labeled with anti-V5) or specifically pro-PC5A (labeled with anti-pro-PC5A).

FIG. 13 illustrates furin siRNA and PC5A siRNA results in 80% knockdown of these convertases in U343 and U251 cells, respectively, wherein: knockdown experiments using siRNAs (Ambion) specific for PC5A or furin were carried out in glioma cells; cells were successfully transfected with siRNA (FIG. 12A), and RT-PCR demonstrated an 80% reduction of furin mRNA levels in U343 cells and PC5 mRNA levels in 0251 cells (FIGS. 12B and C); Furin or PC5 siRNA did not affect PC7 or N-cadherin mRNA levels (FIG. 12B); in addition, GAPDH levels were not affected by furin or PC5 siRNA, but were reduced by a GAPDH-specific siRNA (FIG. 12B); immunocytochemistry also demonstrated a reduction in furin and PC5A levels in U343 and 0251 cells, respectively (FIG. 12D), but there was no reduction in tubulin, nestin or β-catenin expression (FIG. 12D).

FIG. 14 illustrates ProNCAD expression on the surface of invasive tumor cells, wherein: in (A) Western blot analysis of cell surface biotinylated proteins from metastatic prostate (PPC-1, PC3), bladder (JCA-1, T24), squamous cell (NC1-H226), and breast (MDA-MB-436) carcinoma lines is shown; NCAD cytoplasmic antibody detected total NCAD protein, proN antibody detected precursor protein, and ERK (p44/42) antibody was a marker for cytoplasmic proteins; experiments were carried out with 60 μg of total protein and the proportion of surface to total proNCAD was determined, and showed varying levels of surface proNCAD in these carcinoma cells; values are means ±standard error of the mean (SEM); in (B) Western blot analysis of total cell lysates of WM115 VGP and WM266 metastatic melanoma cell lines is shown; NCAD cytoplasmic antibody detected similar levels of total NCAD protein in both WM115 and WM266 melanoma cells, and the proN antibody specifically detected substantially higher levels of proNCAD in WM266 cells; L-cells overexpressing NCAD (LN cells) were used as a control; in (C) Western blot analysis of surface biotinylated U343 and U251 cells is shown; NCAD cytoplasmic antibody detected total NCAD protein, proN antibody detected proNCAD, and ERK (p44/42) antibody was a marker for cytoplasmic proteins; experiments were carried out with 90 or 60 μg of total protein and the proportion of surface to total proNCAD was determined, and found to be approximately three fold higher in U251 cells compared to U343 cells; values are means ±SEM; in (D) immunocytochemistry demonstrating intracellular localization of proNCAD in permeabilized tumor cells (top panels), and substantial surface localization in U251 and WM266 non-permeabilized (bottom panels), live tumor cells is shown; WM115 cells expressed surface proNCAD to a lesser extent than WM266 cells; Bar, 10 μm; in E and F aggregation assay of glioma and melanoma cells, in the presence of calcium is shown; results were quantified as percent of single cells relative to t=0 min, and demonstrate that U343 cells and WM115 cells exhibit high aggregation relative to U251 and WM266 cells, respectively; values are means ±SEM.

FIG. 15 illustrates dependence of migration and aggregation of U251 and U343 cells on furin expression, wherein: in (A) Semi-quantitative RT-PCR was carried out to look at expression of furin, and results show differential expression of furin in U343 and U251 cells, with GAPDH expression used as a normalizing control; real-time PCR was carried out to quantify furin expression in these cells; the results are plotted as mRNA transcripts, normalized with respect to that of ribosomal protein S14, and show contrasting furin expression patterns in U343 and U251 cells; each real-time PCR experiment was carried out in triplicate, and values are means ±SEM; in (B) a wound healing assay was carried out with mock transfected cells, or U251-furin cells; in addition, this assay was carried out with U343 cells transfected with furin siRNA; Bar, 50 μm; the presence of surface proNCAD was also detected in these U343 and U251 transfected cells under non-permeabilizing conditions by immunocytochemistry; Bar, 10 μm; migration was monitored over a 24h period, and results were quantified as number of migrated cells at 12h; values are means ±SEM; in (C) an adhesion assay was carried out with the stably transfected glioma cells, and siRNA transfected cells; cell aggregation was monitored over a 40 min time period and results were quantified as % single cells over time; values are means ±SEM; Bar, 50 μm.

FIG. 16 shoes immunostaining demonstrating tumor formation of glioma cells in vivo, wherein: in (A) U343 glioma cells were transfected with empty vector, wt NCAD-myc, or mutant proNCAD-myc, and injected into the striatum of SCID mice; mice were sacrificed 30 days post-injection, and immunohistochemistry using human nuclei antibody with a hemotoxylin counter stain was performed on fixed brain sections; shown are representative images used for tracings carried out in FIG. 6; U343 cells transfected with wt NCAD-myc formed a solid tumor mass in the striatum of the injected side, but there was no mass detected on the contralateral side; Bar for top panels, 100 μm; Bar for lower panels, 50 μm; in (B) transfected U343 glioma cells were injected into the striatum of SCID mice, and immunohistochemistry was performed on brain sections from mice sacrificed 30 days post-injection; under all transfection conditions, tumor cells stained positive for human nuclei, Ki67 (MIB-1), and myc; however, only U343 transfected with the proNCAD mutant exhibited intense proN staining; double staining was carried out for human nuclei and proNCAD, and single staining was carried out for ki67 and myc; Bar, 25 μm.

FIG. 17 shows that cell surface expression of proNCAD promotes the formation of more aggressive tumors in vivo, wherein: in (A-C) U343 glioma cells were transfected with empty vector, wt NCAD-myc, or mutant proNCAD-myc, and injected into the striatum of SCID mice; mice were sacrificed 30 days post-injection, and immunohistochemistry using an anti-human nuclei antibody with a hemotoxylin counter stain was performed on fixed brain sections; typical three-dimensional reconstructions using the Neurolucida software are shown for each condition; compared to the other conditions, U343-proNCAD-myc cells formed multiple tumor foci and invaded the brain parenchyma in both the injected and non-injected hemispheres as single cells or small groups of cells; red closed contours or markers represent tumors or single cells, respectively, in the injected hemisphere, yellow markers represent cells migrating along the corpus callosum, and blue closed contours or markers represent tumors or single cells, respectively, in the non-injected hemisphere; in (D) immunohistochemistry using the proN antibody was carried out on sections from brains that were injected with U343-proNCAD-myc cells; cells expressing proNCAD were found migrating along ventricles (V), and the corpus callosum (CC) (top panel; Bar, 25 μm; and middle panel, higher magnification; Bar, 15 μm), and throughout the non-injected striatum (S) (bottom panel; Bar, 15 μm); quantification using the Neurolucida software reveals roughly 12 times more single cells invading the brain parenchyma (E), and double the mean invasion distance of single cells from the injection site (F), compared to the other conditions; values are relative to U343-myc, and are means ±SEM of three independent experiments.

FIG. 18 shows cleavage by Factor Xa does not compromise the integrity of mature NCAD, wherein Western blot analysis of total cell lysates of proNCAD-myc or mock transfected cells demonstrates that proNCAD-myc levels are decreased to background upon treatment with the specific protease, Factor Xa, and cleavage with Factor Xa does not compromise the integrity of the mature protein, wherein: in (A) ProNCAD is detected with the proN antibody; in (B) both proNCAD and the mature protein are detected with the NCAD cytoplasmic antibody; in (C) Western blot analysis of conditioned medium collected from proNCAD or mock transfected cells demonstrated specific cleavage of the mutant pro-fragment as an accumulation of the pro-fragment in the medium due to treatment with Factor Xa.

FIG. 19 shows furin siRNA results in 80% knockdown of this convertase in U343 cells, wherein knockdown experiments using siRNAs (Ambion) specific for furin were carried out in glioma cells; cells were successfully transfected with siRNA (A), and RT-PCR demonstrated an 80% reduction of furin mRNA levels in U343 cells (B and C); furin siRNA did not affect PC7 or NCAD mRNA levels (see FIG. 19B), and GAPDH levels were not affected by furin siRNA, but were reduced by a GAPDH-specific siRNA (FIG. 19B); immunocytochemistry also demonstrated a reduction in furin levels in U343 cells, but there was no reduction in tubulin, nestin or β-catenin expression (D); Bar, 10 μm.

FIG. 20 illustrates gross tumor formation of transfected WM266 injected in SCID mice, wherein: WM266 melanoma cells were transfected with empty vector, wt NCAD-myc, or mutant proNCAD-myc, and injected into the intra-peritoneal (IP) cavity of SCID mice; gross inspection of mice injected with WM266-myc cells 30 days post-injection revealed the presence of pigmented subdermal tumors and several polyps associated with the peritoneum or the small intestine, liver, or spleen; mice injected with WM266-wt NCAD-myc were generally found to have smaller or no subdermal tumors, and fewer or no polyps; mice injected with WM266-proNCAD-myc were bloated and developed ascitis, and were found to have numerous polyps associated with the peritoneum, liver, spleen, diaphragm, small and large intestine, and stomach.

FIG. 21 shows an N-cadherin band of slower relative mobility is detected in invasive glioma cells and melanoma cells isolated from a metastatic site, wherein: Western blot analysis of melanoma (WM115 and WM266) and glioma (U343 and U251) total cell lysates is shown; Ncad EC2 Ab detected total Ncad protein.

FIG. 22 shows that ProNCAD is highly expressed on the surface of high grade and metastatic carcinomas, wherein: proNCAD immunoreactivity is negligible in normal brain (A), dermal (B), breast (C), laryngeal (D), and prostate (E) tissues, and expressed at low levels in low grade glioma (A), melanoma (B), breast carcinoma (C), squamous cell carcinoma (D), and prostate carcinoma (E); in contrast, proNCAD expression is strikingly elevated in high grade carcinomas and in corresponding metastases to distant sites (A-E); Bar, 10 μm.

### DETAILED DESCRIPTION OF THE PREFERRED EMBODIMENT

The invention described herein is based, at least in part, on the novel and unexpected observation that cadherin molecules undergo altered proteolytic processing during malignant transformation. This results in a mixture of cadherin molecular forms at the cell surface of cancer cells with altered adhesiveness and functionally enhances cellular migration and invasion.

We report herein our studies of the cellular localization, molecular form and functional state of cell surface cadherin, such as N-cadherin, in cancer cells, such as primary glial tumors and during melanoma transformation. These studies have led to the novel and unexpected finding that in cancer, e.g. during malignant melanoma transformation and in highly invasive glioma cells, in addition to mature N-cadherin (NA), significant amounts of non-adhesive forms (NP) of N-cadherin also appear on the cell surface. These non-adhesive forms comprise uncleaved precursor N-cadherin, as well as a form of N-cadherin where the molecule is cleaved at a second inactivating site, downstream of the Trp2 residue which is known to be required for cadherin mediated adhesion. We have also found that a high ratio of NP/NA can promote detachment, tumor cell migration and invasion.

We further report herein that classical cadherins, such as N-cadherin, possess a range of adhesive strengths, and play a critical role in tumor progression. Moreover, intercellular adhesion can be modulated by surface expression of a non-adhesive CAM.

During malignant transformation, the N-cadherin molecule undergoes altered proteolytic processing. This results in a mixture of N-cadherin molecular forms at the cell surface with varying degrees of adhesiveness. In particular, the precursor N-cadherin can escape proper cleavage and be expressed at the cell surface of, for example, aggressive brain tumor cells, as well as malignant melanoma cell lines and other human carcinoma cell lines. In addition, N-cadherin can be processed at a second inactivating cleavage site, for example in highly invasive brain tumor cells, and in VGP melanoma cells with metastatic potential as well. Precursor N-cadherin at the surface and cleavage at the second site appear to be due to dowregulation of the furin, and upregulation of the PC5A convertase enzymes, respectively. Cadherins which have undergone altered proteolytic processing show reduced adhesiveness compared to normally-processed cadherin and serve to enhance cellular migration and invasion.

In one aspect, the amount of non-adhesive cadherin at the cell surface, e.g. surface proN and functionally inactivated N-cadherin determine the degree of cell invasiveness and metastasis, in later stages of tumor progression. For example, in brain tumor cells, the switch from mature N-cadherin to non-adhesive N-cadherin molecules, mediates detachment from the main tumor mass, and invasion over extensive distances, as demonstrated herein using an in vitro assay. Thus, the switch from E-cadherin to N-cadherin, which has been observed in many tumors, is in many cases a switch to a mixture of N-cadherin molecules where only a certain proportion is functionally adhesive. Similarly, there is a NA to NP switch in brain tumors. In both cases, there is a transition from a functionally adhesive cadherin to one exhibiting compromised adhesion. By altering the cadherin composition at the cell surface, the adhesive strength of nascent cell-cell contacts may be regulated, allowing for fine-tuning of malignant intercellular connections.

Classical cadherins are synthesized as inactive propeptide precursors which become functional mature proteins upon post-translational processing. The proprotein convertases (PCs) are a family of Ca+2-dependent endoproteases responsible for the cleavage of precursor proteins by cleavage at a consensus recognition site. The common mammalian PCs described are furin, PC7, PACE4, PC5, PC⅓, PC2 and PC4. In particular, furin, PC7, PACE4 and PC5 have a wide tissue distribution and proteolytically process precursors in the constitutive secretory pathway. Furin is known to cleave pro-E-cadherin, and precursor N-cadherin, like other classical cadherins, has a consensus cleavage site for PCs at the C-terminal end of the prodomain.

PC5 is expressed as either the A or B isoform. These isoforms are generated by alternative splicing; the B isoform contains all of A except for a small part of its carboxyl-terminus that is positioned after the splice site. The B isoform also includes a transmembrane domain which is not present in the A isoform. PC5 is also known as PC5/6, PC5/6B, PC5A, PC5B, PC5A/B, PC6, PC6A, and PC6B, and these terms are used interchangeably herein. It is contemplated that all forms of the PC5 enzyme are encompassed by the methods and compositions of the present invention. For a review of proprotein convertases, see Thomas, G. (2002) Nat Rev Mol Cell Biol 3:753-766, the entire contents of which are hereby incorporated by reference in their entirety.

It is also provided herein that differential expression of PC enzymes may be a common mechanism in many types of tumors to regulate cellular motility and perhaps other malignant traits, by regulating the processing of cadherins. We report herein that furin is expressed at low levels in invasive tumor cells expressing precursor cadherin at the cell surface. In contrast, expression of PC5, which cleaves N-cadherin at position 28 in EC1, is high in invasive cells relative to non-invasive cells. Cleavage at position 28 in EC1 abolishes the adhesive function of N-cadherin since the Trp2 residue, which is required for adhesiveness, is lost. Therefore both low furin levels and high PC5 levels are correlated with a higher ratio of non-adhesive to adhesive forms of cadherin at the cell surface.

Cancer refers herein to a cluster of cancer or tumor cells showing over-proliferation by non-coordination of the growth and proliferation of cells due to the loss of the differentiation ability of cells. The terms “cancer cell” and “tumor cell” are used interchangeably herein.

The term “cancer” includes but is not limited to, breast cancer, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer (generally considered the same entity as colorectal and large intestinal cancer), fallopian tube carcinoma, endometrial carcinoma, cervical cancer, vulval cancer, squamous cell carcinoma, vaginal carcinoma, Hodgkin's disease, non-Hodgkin'lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer or a combination thereof. In an embodiment, the cancer is a brain tumor, e.g. glioma. The term “cancer” also includes pediatric cancers, including pediatric neoplasms, including leukemia, neuroblastoma, retinoblastoma, glioma, rhabdomyoblastoma, sarcoma and other malignancies.

In a particular embodiment, the invention relates to melanoma, breast cancer, prostate cancer, bladder cancer, squamous cell cancer, and/or brain cancer, such as malignant glioma, such as Glioblastoma multiforme (GBM). In another particular embodiment, the invention relates to epithelial carcinomas.

In another embodiment, the cancer expresses a cadherin protein on the cell surface. In a particular embodiment, the cancer expresses a cadherin protein on the cell surface with altered processing or reduced adhesiveness compared to the cadherin expressed on normal, i.e. non-cancerous cells.

Cadherin proteins which can be used in the methods and compositions of the invention include members of the classical type I and type II cadherin subfamilies. Cadherins are single-pass transmembrane proteins characterized by the presence of distinctive cadherin repeat sequences, consisting of about 110 amino acids, in their extracellular segments. Cadherins can be classified into several subfamilies based on shared properties and sequence similarity. Classical (type I) cadherins have a conserved tryptophan at position 2 of the mature protein, which is a central feature of the cell-cell adhesive interface. The pre- or pro-domain must be removed by furin family proteases for these molecules to mediate functional adhesion. Type II cadherins are different from type I cadherins in that they have a smaller pre- or pro-domain and two conserved tryptophan residues in their EC1 domain. Both type I and type II cadherins are linked to the actin cytoskeleton through specific adaptor proteins. For a review of cadherin proteins, see Nollet et al., J. Mol. Biol. (2000) 299: 551-572, and Patel et al., Curr. Opin. in Struct. Biol. (2003) 13: 690-698, the entire contents of which are hereby incorporated by reference.

Non-limiting examples of type I and type II cadherins which can be used in the methods and compositions of the invention include E-cadherin (also known as uvomorulin, L-CAM and cadherin-1), N-cadherin (also known as cadherin-2), C-cadherin, R-cadherin (also known as XmN-cadherin and cadherin-4), VE-cadherin (also known as cadherin-5), K-cadherin (also known as cadherin-6), T1-cadherin (also known as cadherin-9), T2-cadherin (also known as cadherin-10), OB-cadherin (also known as cadherin-11), Br-cadherin (also known as N-cadherin-2, cadherin-12, M-cadherin (also known as cadherin-15), P-cadherin, Cadherin-14 (also known as cadherin-18 or mouse EY-cadherin), cadherin-7, F-cadherin (also known as cadherin-20), cadherin-8, cadherin-19, EP-cadherin (XI), BS-cadherin (Bs) and PB-cadherin (Rn). It is contemplated that any cadherin which undergoes proteolytic processing, and for which the adhesiveness of the processed form differs from that of the unprocessed form, is encompassed by the invention described herein and can be used in the methods and compositions of the invention.

In accordance with the present invention, there is provided a method for diagnosing cancer and determining prognosis in a subject by characterizing the molecular form of cadherin expressed at the cell surface of cancer cells in the subject.

In one aspect of the invention, there is a provided a method of diagnosing and/or determining the prognosis of a cancer by determining the molecular forms of cadherin at the cell surface of the cancer cells. In one embodiment, anon-adhesive form of cadherin at the cell surface is diagnostic of a more aggressive, or more highly invasive, tumor. In another embodiment, the non-adhesive form of cadherin is the precursor or “pro-” form. In another embodiment, the non-adhesive form has been cleaved before the Trp2 residue. In yet another embodiment, the non-adhesive form has been cleaved by the PC5 convertase. Any form of cadherin which is non-adhesive or has reduced adhesiveness compared to normally-expressed cadherin is encompassed by the methods herein. Detection of non-adhesive cadherin, e.g. proN-cadherin, at the cell-surface in a cancer cell may therefore serve as a diagnostic and/or prognostic tool for staging and progression of the disease.

The terms “proNCAD”, “precursor N-cadherin”, “proN-cadherin” and “proN” are used interchangeably herein, and refer to the precursor or “pro-” form of N cadherin, i.e. the form of N-cadherin which has not been cleaved by furin and contains the pro domain. The terms “N-cadherin” and “NCAD” are used interchangeably herein and refer to N-cadherin (also known as cadherin-2). Similar terminology is used for the other cadherins, for example E-cadherin is also referred to as ECAD.

In another embodiment, a high ratio of non-adhesive to adhesive cadherin forms at the cell surface is diagnostic of a more aggressive, or highly invasive tumor. In another aspect, the invention provides methods of monitoring the progression of a cancer and/or monitoring the efficacy of an anti-cancer treatment or therapeutic regimen. It is contemplated that any anti-cancer treatment or therapeutic regimen known in the art could be used in the methods described herein. Non-limiting examples of treatments and therapeutic regimens encompassed herein include surgery, radiology, chemotherapy, and administration of targeted cancer therapies and treatments, which interfere with specific mechanisms involved in carcinogenesis and tumour growth.

Non-limiting examples of targeted cancer therapies include therapies that inhibit tyrosine kinase associated targets (such as Iressa®, Tarceva® and Gleevec®), inhibitors of extracellular receptor binding sites for hormones, cytokines, and growth factors (Herceptin®, Erbitux®), proteasome inhibitors (Velcade®) and stimulators of apoptosis (Genasense®). Such targeted therapies can be achieved via small molecules, monoclonal antibodies, antisense, siRNA, aptamers and gene therapy. A subject may also receive a combination of treatments or therapeutic regimens. Any other treatment or therapeutic regimen known in the art can be used in the methods described herein, alone or in combination with other treatments or therapeutic regimens.

In one aspect of the invention, therefore, the invention provides methods of monitoring the progression of a cancer and/or monitoring the efficacy of an anti-cancer treatment or therapeutic regimen by determining the molecular forms of cadherin at the cell surface of cancer cells. In one embodiment, a non-adhesive form of cadherin at the cell surface or a high ratio of non-adhesive to adhesive cadherin forms indicates that a cancer has progressed to an invasive, metastatic phase. In another embodiment, a subsequent decrease in the ratio of non-adhesive to adhesive cadherin in a cancer cell indicates further progression of the cancer to a less invasive stage.

In one embodiment, the non-adhesive form of cadherin is the precursor or “pro-” form. In another embodiment, the non-adhesive form has been cleaved before the Trp2 residue, e.g. by the PC5 convertase.

The molecular form of cadherin at the cell surface may be determined using standard methods known in the art. In one aspect, the molecular form of cadherin at the cell surface is determined in a sample from a subject, e.g. a tissue sample obtained via biopsy. Non-limiting examples of such methods include immunodiagnostic methods such as immunohistochemistry, immunocytochemistry, western blotting, radioimmune assay (RIA) and so on. In an embodiment, the molecular form is determined using an antibody specific for a particular molecular form, e.g. an antibody specific for the pro-domain, e.g. anti-proN, or an antibody specific for a particular cleavage form. In another aspect, the cadherin may be analyzed in a subject directly using imaging techniques known in the art such as radionuclide imaging, SPECT imaging, magnetic resonance imaging, fluorescence imaging, positron emission tomography, CT imaging, or a combination thereof. In one aspect, the cadherin may be analyzed in a subject directly using a detectably-labeled antibody, e.g. a detectably-labeled anti-proN antibody.

In another aspect of the invention, there is a provided a method of diagnosing and/or determining the prognosis of a cancer in a subject by determining the levels of expression of preprotein convertases in cancer cells. In one embodiment, the level of expression of furin is determined. In another embodiment, the level of expression of the PC5 convertase is determined. Low expression levels of furin correlate with expression of the precursor form of cadherin at the cell surface and are therefore diagnostic of a more aggressive, highly invasive tumor. High levels of PC5 convertase correlate with presence of a non-adhesive cleavage form of cadherin at the cell surface and are therefore diagnostic of a more aggressive, highly invasive tumor. In an aspect, expression levels of one or more than one convertase may be determined. For example, low furin expression levels and/or high PC5 expression levels is indicative of an invasive tumor, wherease high furin and/or low PC5 levels indicate a non-invasive tumor. Convertase levels may be determined alone or in combination. It is contemplated that expression levels of any proprotein convertase enzyme which cleaves a cadherin and thereby modulates its functional adhesiveness may be used in the methods of the invention.

Convertase expression levels may be determined using standard methods known in the art. Non-limiting examples of such methods include immunoblotting, methods to determine mRNA levels such as RT-PCR and northern analysis, real-time PCR, PCR, immunocytochemistry, immunohistochemistry, radioimmune assay (RIA), and so on.

In another aspect of the invention, the invention provides methods of monitoring the progression of a cancer and/or monitoring the efficacy of an anti-cancer treatment or therapeutic regimen by determining the levels of expression of proprotein convertase enzymes in cancer cells. In one embodiment, a low level of furin expression and/or a high level of PC5 expression indicates that a cancer has progressed to an invasive, metastatic phase. In another embodiment, a subsequent increase in furin expression and/or decrease in PC5 expression in a cancer cell indicates further progression of the cancer to a less invasive stage.

In yet another aspect, the invention provides a method of assigning an anti-cancer treatment or a therapeutic regimen to a subject. In one aspect, the method comprises determining the molecular forms of cadherin at the cell surface of the cancer cells in a subject, wherein a non-adhesive form of cadherin at the cell surface or a high ratio of non-adhesive to adhesive cadherin forms indicates that a cancer has progressed to an invasive, metastatic phase, and treatment appropriate for an invasive, metastatic cancer is assigned accordingly. In another embodiment, a subsequent decrease in the ratio of non-adhesive cadherin to adhesive cadherin in a cancer cell indicates further progression of the cancer to a less invasive stage and treatment may be modified accordingly. In another embodiment, the levels of expression of proprotein convertase enzymes in the cancer cells in a subject are determined, wherein a low level of furin expression and/or a high level of PC5 expression indicates that a cancer has progressed to an invasive, metastatic phase, and treatment is assigned accordingly. In another embodiment, a subsequent increase in furin expression and/or a decrease in PC5 expression in a cancer cell indicates further progression of the cancer to a less invasive stage and treatment is modified accordingly.

Kits for diagnosing or determining prognosis of a cancer in a subject, comprising reagents for determining the molecular form of cadherin at the cell surface of cancer cells in the subject, and instructions for use thereof, are also provided herein. The reagents may comprise one or more than one probe capable of detecting non-adhesive forms of cadherin at the cell surface, e.g. an antibody binding specifically to a non-adhesive form such as the pro-form or a cleavage form. In one aspect, the antibody may be specific for the pro-domain (also referred to as the pro-region) of a cadherin. In another aspect, the antibody may be specific for the pro-domain of N-cadherin. In another aspect, the antibody may be anti-proN (Koch et al. (2004) Structure 12: 793-805). The reagents may also comprise probes binding specifically to cadherin mRNA, e.g. N-cadherin mRNA, to allow detection of expression of e.g. N-cadherin. Kits for diagnosing or determining prognosis of a cancer in a subject, comprising reagents for determining expression levels of one or more than one proprotein convertase, e.g. furin or PC5, in cancer cells in the subject, and instructions for use thereof are also provided. The reagents may comprise, for example, PCR reagents, primers specifically hybridizing to proprotein convertase mRNA or a fragment thereof, antibodies specific for a proprotein convertase, e.g. furin and/or PC5, and/or reagents for assaying furin or PC5 enzymatic activity.

In a further aspect of the invention, there is provided a method for preventing, inhibiting, or treating cancer and/or the metastasis or spread thereof by decreasing the amount of non-adhesive cadherin forms at the cell surface of a cancer cell, by increasing the amount of adhesive cadherin forms at the cell surface of a cancer cell, or by decreasing the ratio of non-adhesive to adhesive cadherin forms at the cell surface of a cancer cell. In one aspect, the expression or activity of a proprotein convertase, e.g. furin, is increased. In another aspect, expression or activity of a proprotein convertase, such as PC5, is inhibited, e.g. by administration of an inhibitor.

In one aspect, an effective amount of a proprotein convertase inhibitor is administered to a subject to prevent, inhibit, or treat cancer and/or the metastasis or spread thereof by e.g. decreasing the amount of non-adhesive cadherin forms at the cell surface of a cancer cell, or increasing the amount of adhesive cadherin forms at the cell surface of a cancer cell, or decreasing the ratio of non-adhesive to adhesive cadherin forms at the cell surface of a cancer cell. In an aspect, the inhibitor may be e.g. decanoyl-RVKR-chloromethylketone or an alpha-1-antitrypsin variant, e.g. alpha-1-PDX (see, for example, Jean et al. (1998) Proc. Natl. Acad. Sci. USA 95:7293-7298; Tsuji et al. (2007) Protein Eng Des Sel 20: 163-170; the entire contents of which are hereby incorporated by reference). It is contemplated that PC5 inhibitors known in the art may be used in the methods and compositions of the invention. In one aspect, a PC5 inhibitor may be administered to a subject in need thereof. In another aspect, the amount of adhesive forms of cadherin at the cell surface may be increased by adding furin to the surface of a tumor. In another aspect, furin may be administered to a subject in need thereof. In another aspect, PC5 levels may be inhibited or decreased using antisense RNA or siRNA.

The present invention will be more readily understood by referring to the following examples, which are given to illustrate the invention rather than to limit its scope.

### Example 1

### Human Glioma and Melanoma Cells Express Functionally Adhesive N-cadherin

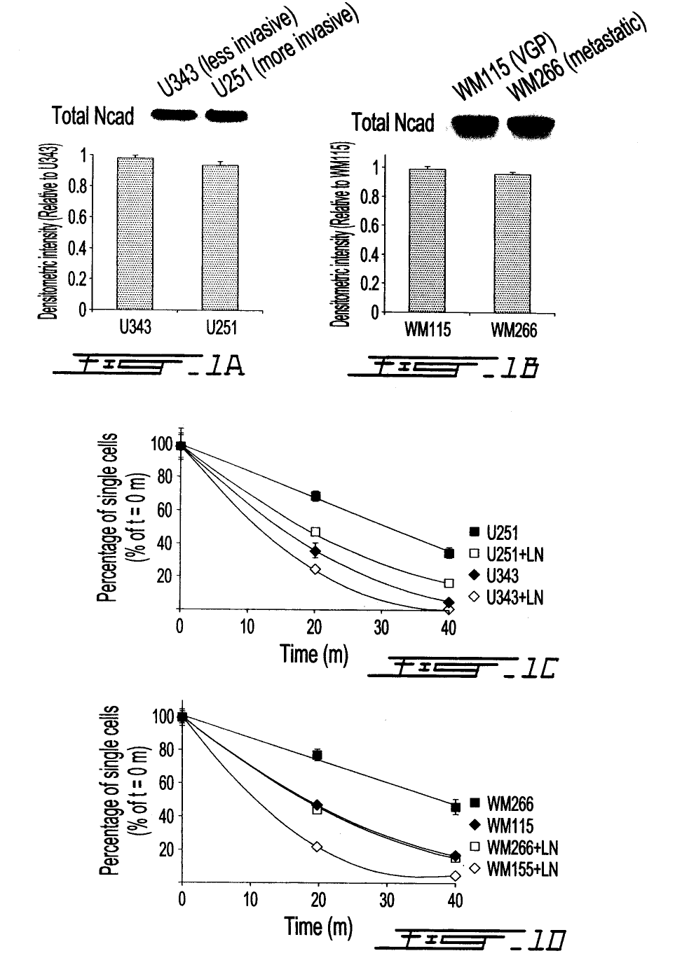

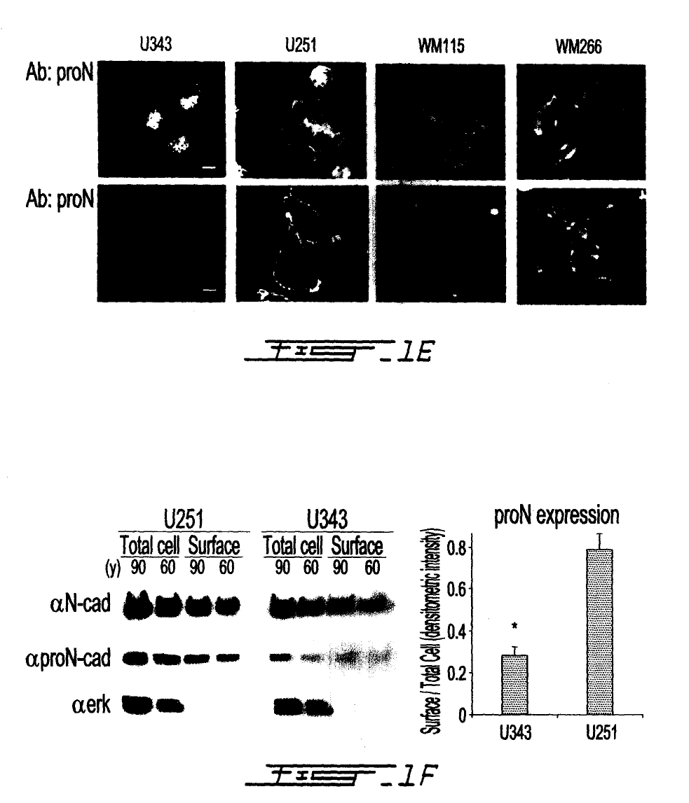

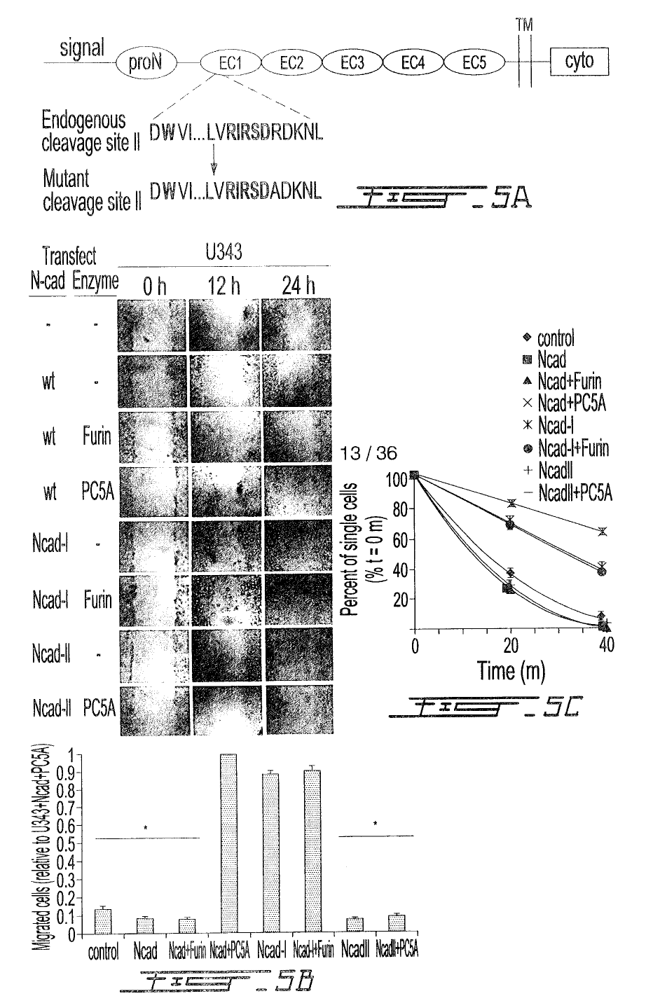

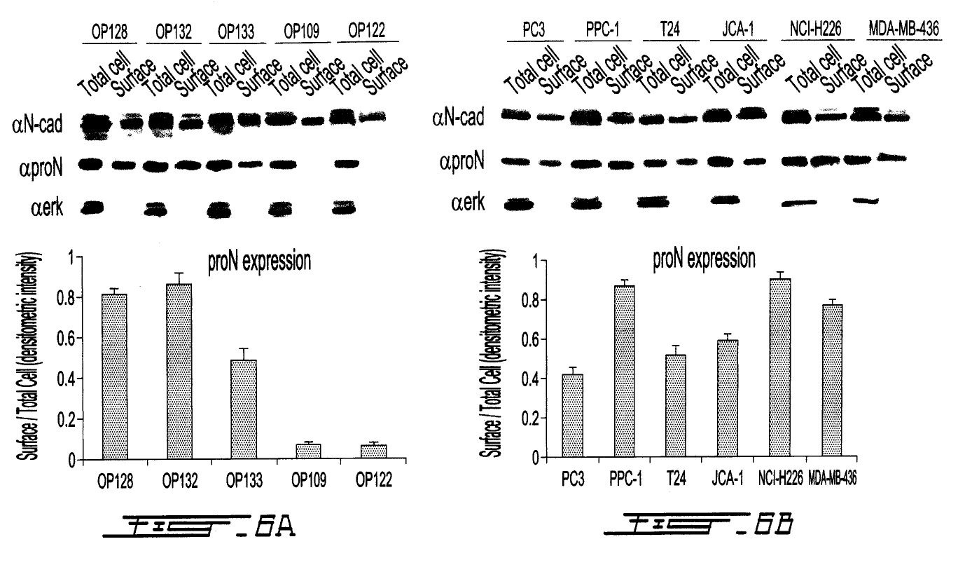

We studied the functional state of surface expressed N-cadherin in primary malignant glioma cell lines, and in melanoma cell lines representing different stages of transformation. N-cadherin expression was comparable in U343 and U251 cell lines (FIG. 1A), which invade approximately 500 μm and 1400 μm, respectively, in a three-dimensional invasion assay 5 days post-implantation (FIG. 8), as well as in VGP (WM115) melanoma cells and a melanoma cell line established from a secondary site (FIG. 1B).

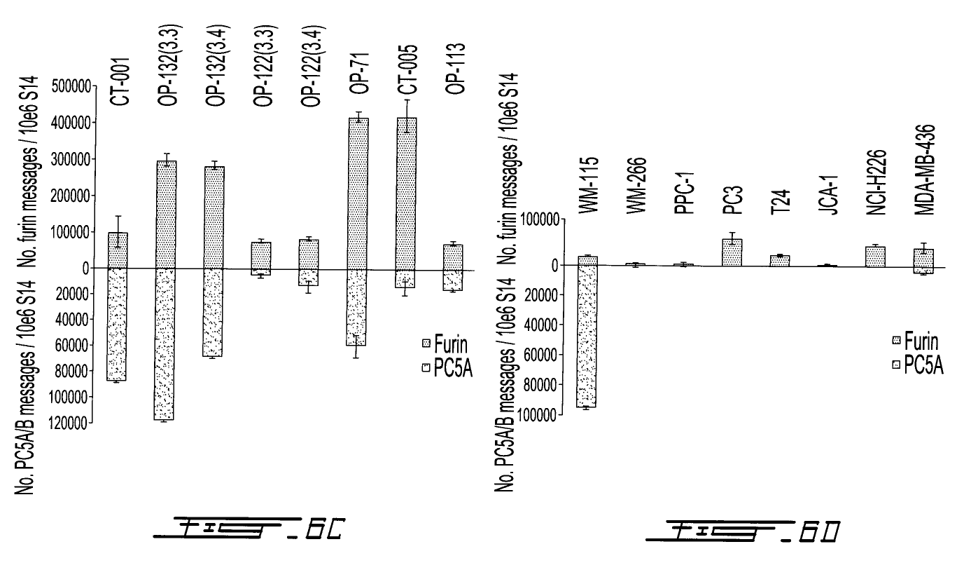

Since N-cadherin is an abundant component of melanoma and glioma cell lines, we wanted to examine its adhesive activity in these cells. We observed greater aggregation in less invasive U343 glioma cells and in VGP cells (WM115), compared to highly invasive U251 cells and metastatic melanoma cells (WM266), respectively (FIG. 1, C and D; FIG. 9A). There was no cell aggregation in the absence of calcium for all cell lines (data not shown), revealing that calcium-dependent cadherin mediated adhesion is the only adhesion mechanism of consequence in these cell lines. Aggregation assays were carried out by mixing either tumor cell lines (labelled with Dil) with L cells overexpressing N- or E-cadherin (LE cells or LN cells, labeled with DiO). We observed mutually exclusive segregation of tumor cells from LE cells, and co-aggregation of tumor cells with LN cells (FIG. 9B). Together, these results reveal that in these aggregation assays, N-cadherin is a primary mediator of adhesion in both melanoma and glioma cells.

Cell surface Expression of Precursor N-cadherin Promotes Motility of Glioma and Melanoma Cells

Aggregation was notably faster in experiments where LN, cells were mixed with tumor cells, as compared with experiments with tumor cells alone (FIG. 1, C and D). This was especially pronounced for the WM266 and U251 lines, where there was −30% less single cells at 20 min when mixed with LN cells. In addition, we consistently noted a band of slower relative mobility than that of mature N-cadherin, consistent with an N-cadherin molecule in which removal of the N-terminal pro-piece did not occur efficiently, resulting in retention of the prodomain(proN Mr135 kDa, Cad Mr120 kDa; data not shown). This slower band represented between ˜20%-60% of the total N-cadherin protein on these blots, and it was of great interest that glioma cells with a higher invasion potential, and melanoma cells isolated from a metastatic site exhibited higher levels of the band.

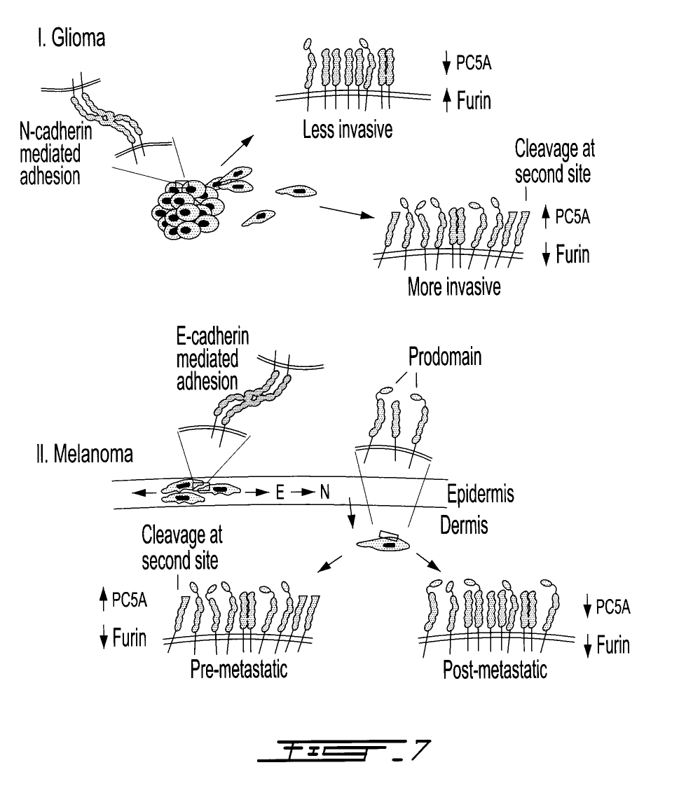

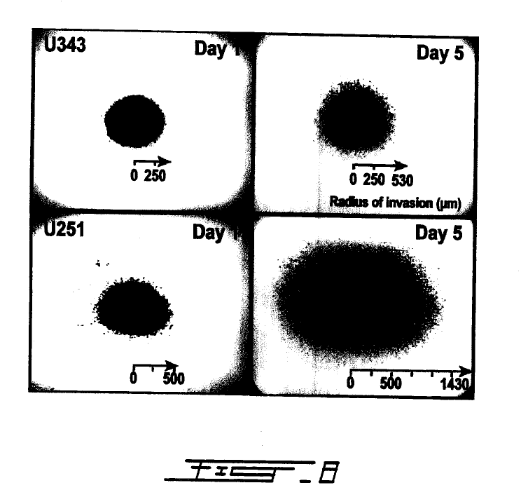

To investigate this further, we generated a rabbit polyclonal antibody specifically against the N-cadherin prodomain (anti-proN). Using this antibody, we looked at immunolocalization of the N-cadherin precursor (proN) and found that it could be detected intracellularly in permeabilized glioma and melanoma cells (FIG. 1E, top panels). This is in agreement with the fact that proN is normally intracellular (Wahl et al. (2003) J Biol Chem 278; 17269-17276), and was never known to be on the cell surface. Surprisingly, we found that proN was also detected on the cell surface of non-permeabilized, live U251 and WM266 cells, and to a much lesser extent on the surface of WM115 cells (FIG. 1E, bottom panels), and co-existed with mature N-cadherin (FIG. 10). proN was not on the surface of U343 cells (FIG. 1E, bottom panels). We then carried out cell surface biotinylation experiments to quantify the proportion of surface expressed proN. We found that a high proportion (80%) of proN was present on the surface of the highly invasive U251 cells, but not of U343 cells (FIG. 1F). Together, these results reveal that even in the presence of mature, adhesively active N-cadherin, cell-surface accumulation of proN is important for migration and invasion.

### Example 2

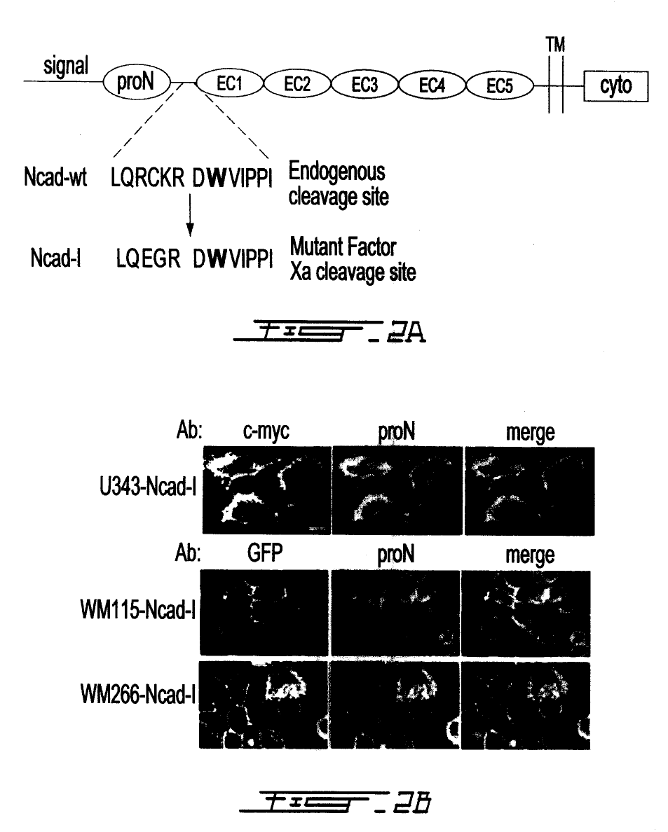

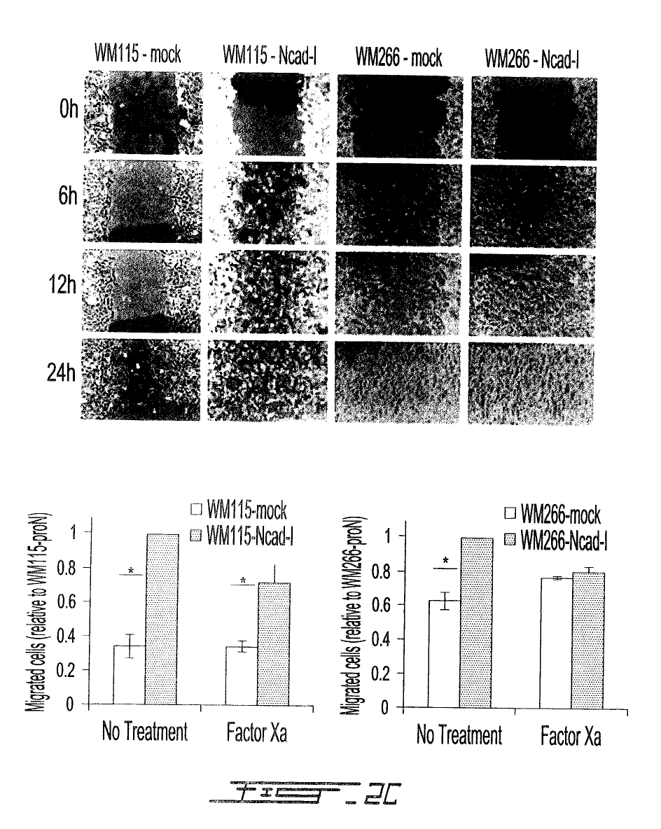

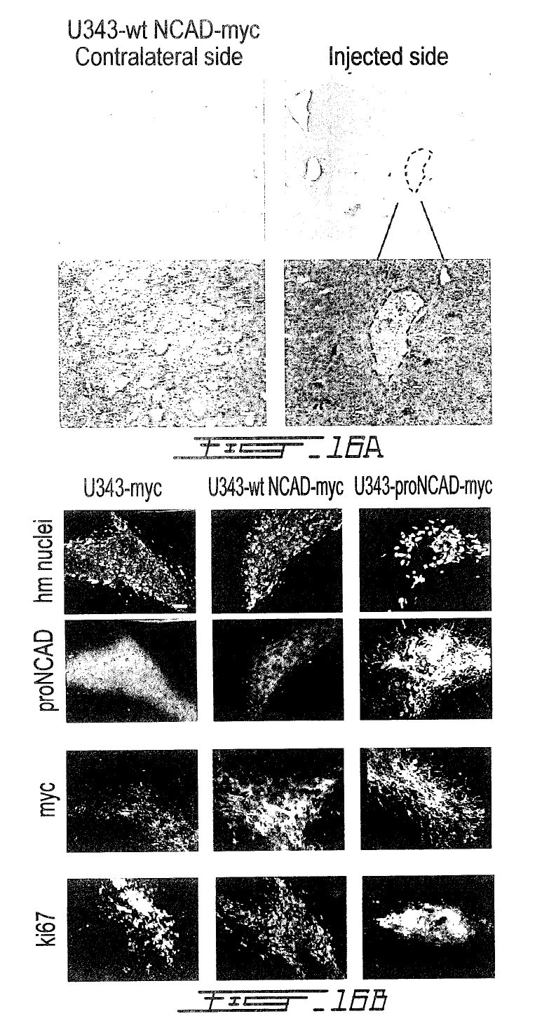

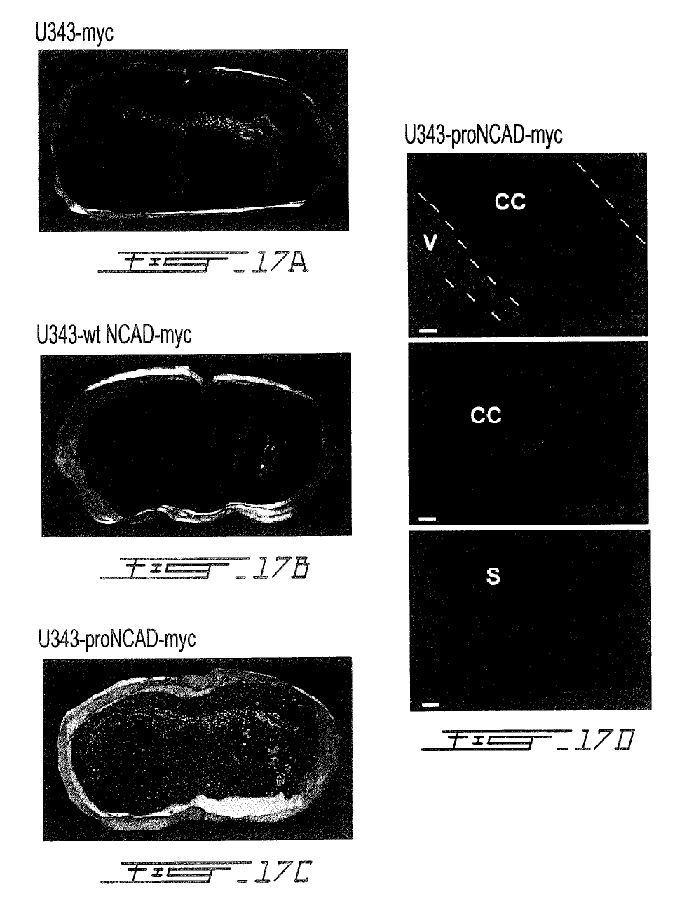

Since N-cadherin expression has been shown to correlate with increased motility and proN lacks adhesive function, we hypothesized that loss of adhesion due to aberrant surface expression of proN may serve as a mechanism for enhanced motility in brain tumor cells, even in the presence of mature N-cadherin. In this way proN could influence for example glioma invasion and melanoma metastasis. We engineered an N-cadherin construct (called Ncad-1, which expresses a mutant precursor protein referred to as Ncad-1 or mutant proNCAD) where the endogenous consensus proprotein convertase cleavage site was replaced with a serum coagulation Factor Xa recognition site in the linker sequence (FIG. 2A), similar to previously reported constructs. Glioma and melanoma cells transfected with mutant Ncad-1-GFP or mutant Ncad-1-myc, respectively, were selected for and clonal populations were expanded. Myc and proN co-localized extensively at the plasma membrane of transfected glioma cells, and GFP and proN showed a similar localization in transfected melanoma cells (FIG. 2B). Western blot analysis of total cell lysates or conditioned medium of transfected cells was carried out to look at cleavage of the prodomain by factor Xa. ProNCAD-myc levels were decreased to background upon treatment with Factor Xa (FIG. 18A), and cleavage did not compromise the integrity of the mature protein, since it ran at 125 kDa similar to NCAD in mock transfected cells (FIG. 18B). Specific cleavage of the mutant prodomain by Factor Xa is demonstrated as accumulation of the 17 kDa fragment in the conditioned medium (FIG. 18C).

To examine the role of proNCAD in cell motility, we performed a wound healing assay in which confluent monolayers are disrupted by scraping with a fine pipette tip. WM115 and WM266 mock transfected cells exhibited reduced migration into the wound compared to WM115 and WM266 cells transfected with mutant proNCAD (FIG. 2C). This effect was abolished upon treatment of proNCAD transfected cells with Factor Xa (FIG. 2C). WM115 cells, which express low levels of surface proNCAD, did not form a confluent cell monolayer after 24h (FIG. 2C). In contrast, WM266 cells re-organized into a fairly dense cell monolayer after 24h. The most dense cell monolayer was observed with WM266 cells transfected with mutant proNCAD, as these cells express both transfected and endogenous cell surface proNCAD (FIG. 2C). Similar to the melanoma cells, U343 glioma cells transfected with mutant proNCAD exhibited increased migration into the wound compared to mock transfected cells, and this effect was abolished upon treatment with Factor Xa (data not shown).

The effect of surface proNCAD on invasion was assessed in three-dimensional collagen invasion assays and Boyden chamber assays. Spheroids of mutant proNCAD or mock transfected glioma cells were implanted into a collagen matrix with or without Factor Xa, and invasion was monitored over 5 days (FIG. 2D). Invasion was quantified on day 3 and demonstrates that compared to the control, transfection with the mutant proNCAD construct resulted in more than double the number of cells invading at distances up to 300 μm from the edge of the spheroid. This effect was most pronounced at distances greater than 300 μm, where there were 10 fold more proNCAD transfected cells invading compared to control cells (FIG. 2D). This effect was reversed upon treatment with Factor Xa. Boyden chamber assays were also carried out to assess the effect of proNCAD on invasion. Cells were seeded in the upper chamber of a Matrigel coated filter, and NIH3T3 cell conditioned medium was used as a chemoattractant in the lower chamber. ProNCAD surface expression substantially increased invasiveness relative to the parental cell lines for both WM115 and WM266 cells, and WM115 cells exhibited a lower rate of invasion compared to WM266 cells (data not shown). Treatment with Factor Xa reduced invasion to levels observed with parental cell lines. Thus, it appears that inhibition of cell-to-cell adhesion by surface-expressed proNCAD promotes malignant tumor cell behaviors such as migration and invasion in glioma and melanoma cells.

### Example 3

### Furin and PC5 Proprotein Convertases are Differentially Expressed in Glioma Cells

Classical cadherins are synthesized as inactive propeptide precursors, which become functional mature proteins upon post-translational processing. The subtilisin-like proprotein convertases (PCs) are a family of Ca2+-dependent endoproteases, responsible for the activation of precursor proteins by cleavage at a consensus recognition site (Arg/Lys-(X)n-Lys/Arg-Arg, n=0, 2, 4 or 6) (Seidah and Chretien (1997) Curr opin Biotechnol;, 602-607). The common mammalian PCs described are furin, PC7, PACE4, PC5, PC1/3, PC2, and PC4. While PC1 and PC2 are important in the endocrine pathway, and PC4 only functions in germinal cells, furin, PC7, PACE4, and PC5 have a wide tissue distribution and proteolytically process precursors in the constitutive secretory pathway. It has been shown that furin can cleave pro-E-cadherin (Posthaus et al. (1998) FEBS Lett 438; 306-310), rendering the molecule functionally adhesive, and precursor N-cadherin, like other classical cadherins, has a consensus cleavage site for PCs (Koch et al. (2004) Structure 12: 793-805; Posthaus et al. (1998) FEBS Lett 438; 306-310) at the C-terminal end of the prodomain. PC5 is expressed as either the A or B isoform. The reagents used herein do not distinguish between these isoforms and the terms PC5, PC5A, PC5B, PC5/6, and PC5/6B are used interchangeably herein.

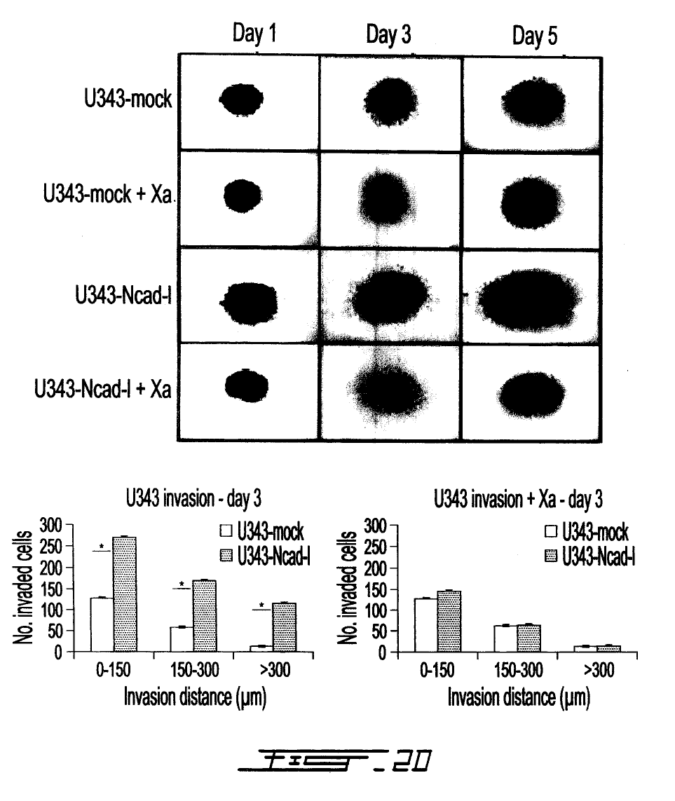

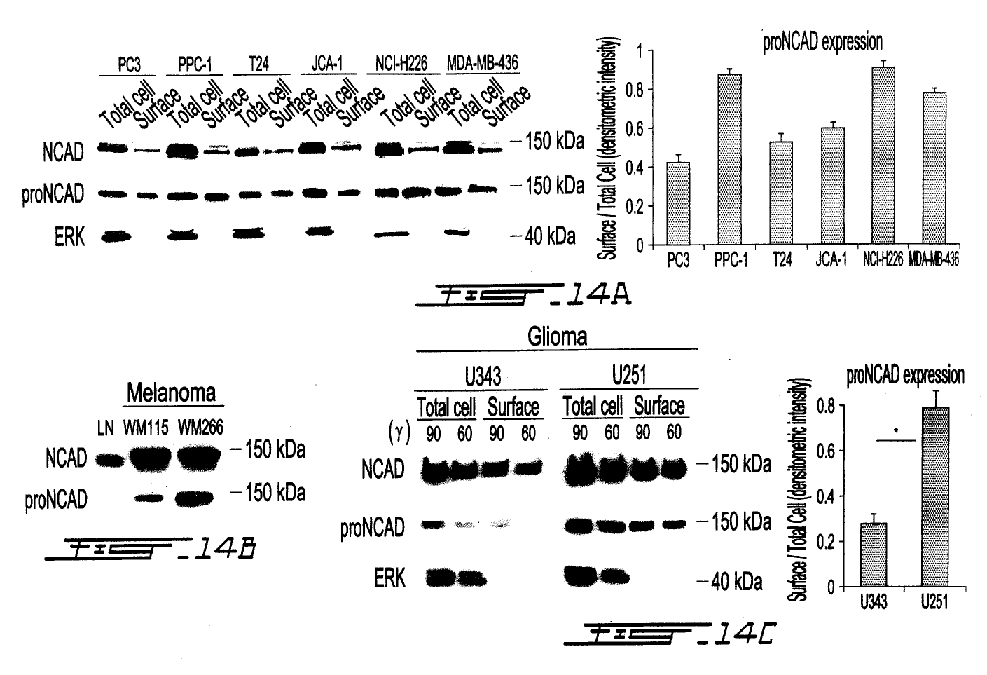

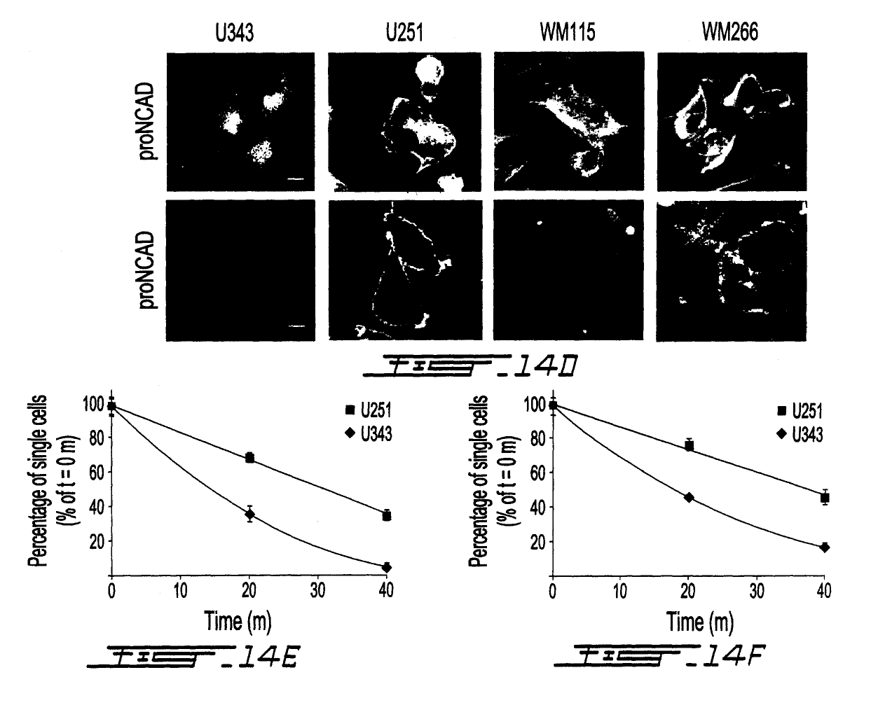

We therefore looked at the expression of furin, PC7, PACE4 and PC5 in the tumor cells to determine whether differences in levels of these enzymes might underlie the mechanism leading to surface expression of proN (also referred to as proNCAD or precursor N-cadherin; these terms are used interchangeably herein). Since only 45% of the highly invasive glioma cells, compared to 70% of metastatic melanoma cells expressed cell surface proN, this suggested that there may be additional mechanisms associated with malignant glioma cell invasion. Semiquantitative RT-PCR revealed similar levels of PC7 in U343 and U251 cells, and expression of PACE4 was not detected in either cell line (FIG. 3A). Interestingly, expression of furin was lower in invasive U251 cells relative to U343 cells, and in contrast to this, PC5 expression was high in U251 cells relative to U343 cells. This difference was quantified by real-time PCR and we found the number of mRNA messages of furin to be 180000/10⁶ S14 RNA transcripts for U343 cells and 5000/10⁶ S14 RNA transcripts for U251 cells, and the number of PC5 messages to be 5000/10⁶ S14 RNA transcripts for U343 cells and 20000/10⁶ S14 RNA transcripts for U251 cells (FIGS. 3B and 15A) by quantitative real-time PCR. Low furin expression in U251 cells was a conceivable explanation for precursor N-cadherin being present on the surface of these cells. Relatively high furin levels would be expected to render tumor cells less invasive and more adhesive to one another since N-cadherin would be properly cleaved at the consensus site, and the contrary would be true for highly invasive brain tumor cells expressing low furin levels.

The contrasting expression of PC5 was quite intriguing, especially since it was demonstrated that E-cadherin was processed in furin-deficient LoVo cells indicating that another convertase can also process N-cadherin (Posthaus et al. (1998) FEBS Lett 438; 306-310). This led us to inspect the N-cadherin sequence for another PC cleavage site. We identified a strong putative site for PC5 or PACE4 where the molecule would be cleaved at position 28 in EC1, downstream of the consensus site (see FIG. 3B). Cleavage at this second site would abolish the adhesive function of N-cadherin since Trp2 would be lost, and thus this would present a mechanism to permanently inactivate the molecule. This second cleavage site in N-cadherin is conserved in many species including human, rat, and mouse.

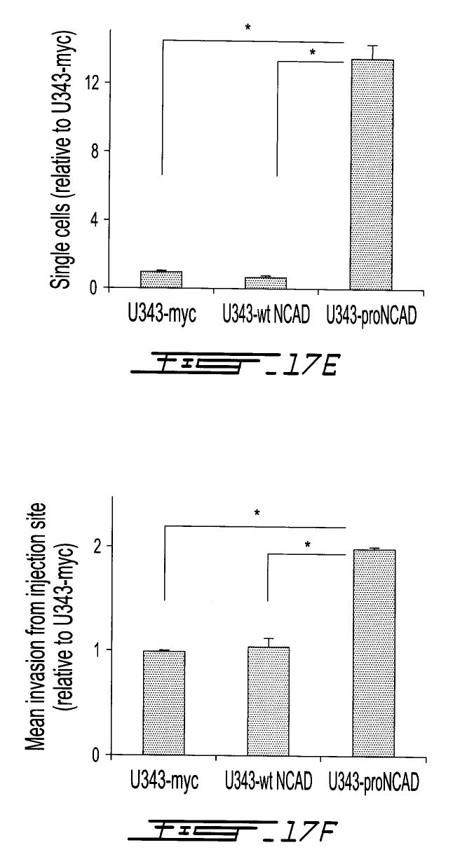

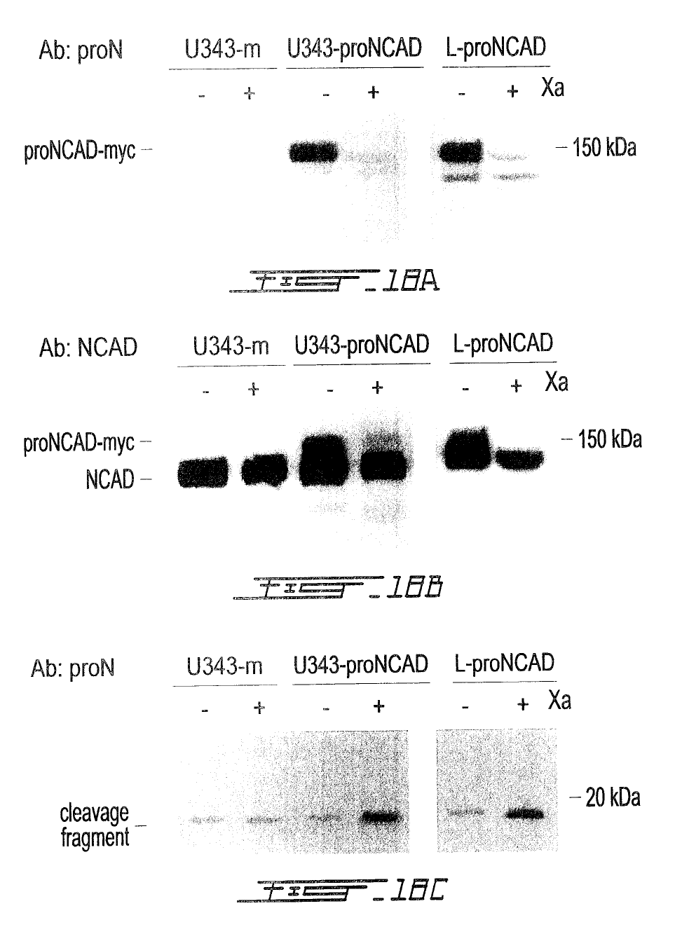

To determine the effects on cellular behavior by furin we carried out knockdown experiments in U343 cells using siRNAs specific for furin and gain of function experiments where U251 cells were stably transfected with furin. Cells were successfully transfected with siRNA (FIG. 19A), and semi-quantitative RT-PCR demonstrated an 80% reduction of furin mRNA levels in U343 cells (FIGS. 19B and 19C). Furin siRNA did not affect PC7 or NCAD mRNA levels (FIG. 19B). In addition, GAPDH levels were not affected by furin siRNA, but were reduced by a GAPDH-specific siRNA (FIG. 19B). Immunocytochemistry also demonstrated a reduction in furin levels in U343 cells (FIG. 19D), but there was no reduction in tubulin, nestin or β-catenin expression (FIG. 19D). Our siRNA results show that compared to control siRNA, knockdown of furin in U343 cells resulted in a substantial increase in cell migration (FIG. 15B), and a decrease in cell aggregation (FIG. 15C). Knockdown of furin also resulted in an increase in surface proNCAD levels under non-permeabilizing conditions (FIG. 15B). In our gain of function studies, transfected cells exhibited colocalization of furin with EGFP (data not shown). Our results show that compared to mock transfections, overexpression of furin in U251 cells resulted in a substantial decrease in the number of migrated cells at 12h in a wound healing assay (FIG. 15B), and in a decrease in surface proNCAD levels under non-permeabilizing conditions (FIG. 15B). U251 cells transfected with furin aggregated to a much greater extent compared to control cells (FIG. 15C). These results demonstrate that furin expression appears to inhibit glioma cell migration by affecting cell-to-cell adhesion.

### Cell Surface PC5Cleaves N-Cadherin at a Second, Inactivating Site

To determine whether N-cadherin can be cleaved at the second site and by which convertase, we carried out a series of transient co-transfections in HeLa cells, which are deficient for PC5A (Essalmani et al. (2006) Molec Cell Biol 26; 354-361). In these experiments, we were able to detect whether N-cadherin was cleaved at the first or second site by identifying cleavage peptides in the conditioned medium using the proN antibody. Our results demonstrate that two cleavage products, one at 17 kDa, and one at 20 kDa are detected when HeLa cells are transfected with N-cadherin and full length (FL) PC5A (FIG. 3C, lane 1). These Mr's are consistent with peptides resulting from cleavage at the consensus site, and at the second site, respectively. The higher Mr cleavage product was not detected when N-cadherin was transfected without PC5A (FIG. 3C, lane 2), or in the vector control (FIG. 3C, lane 4). There were no cleavage products detected in the presence of the PC-specific inhibitor, dec-cmk (Jean et al. (1998) Molec Cell Biol 26, 354-361); FIG. 3C, lanes 5-8). As a positive control for PC inhibition by dec-cmk (Siegfried et al. (2003) Cancer Res 63; 1458-1463), HeLa cells were transfected with propDGF-A and cells were incubated in the absence or presence of 50 μM dec-cmk (right panel). In experiments where PC5A with a deleted cysteine-rich domain (ACRD) was transfected instead of FL-PC5A, the 17 kDa cleavage product was only detected (FIG. 3D). Similar experiments were carried out with PACE4, furin, and PC7. We found that N-cadherin was only cleaved at the consensus site when FL-PACE4 or PACE4-ΔCRD was transfected (FIGS. 3E and 3F), or when furin or PC7 was transfected (FIGS. 3G and 3H). In addition, the 17 kDa product was noticeably more intense when furin was transfected (FIG. 3G, lane 1 vs. lane 3), but not when PC7 was transfected (FIG. 3H, lane 1 vs. lane 3).

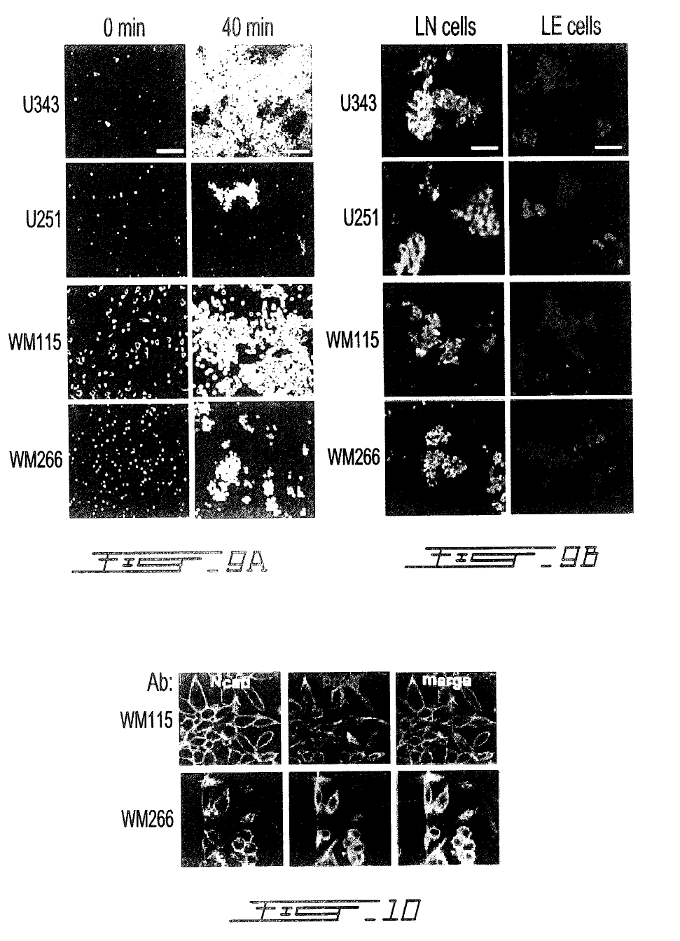

Transfection of HeLa cells with N-cadherin and convertase constructs was verified by running Western blots of total cell lysates using a myc (9E10) antibody to detect N-cadherin expression, and a V5 antibody to detect convertase expression (see FIG. 11, A-F). These results show that PC5A can cleave N-cadherin at the second inactivating site, but only with an intact CRD. This indicates that cleavage at this site likely takes place at the plasma membrane, since it has been previously shown that the CRD domain of PC5A (and PACE4) mediates cell surface anchoring of the enzyme via interactions with heparan sulfate proteoglycans (HSPGs) and the TIMP molecule (Nour et al. (2005) Molec Biol Cell 16; 5215-5226). PACE4, furin or PC7 cannot cleave N-cadherin at this site, and it appears that furin is important for cleavage at the consensus site.

We then looked at localization of PC5A in transfected HeLa cells. PC5A is localized to the cell surface in cells that are transfected with FL-PC5A and stained under non-permeabilizing conditions (FIG. 31). However, it is not detected on the surface of cells if heparin was added to the culture medium (FIG. 31), or in cells stained under permeabilizing conditions (FIG. 31). In addition, PC5A-ΔCRD did not localize to the cell surface (FIG. 31). Thus FL-PC5A convertase localizes to the cell surface of HeLa cells and is able to cleave N-cadherin at the inactivating site. Mechanistically, our data shows that expression levels of furin and PC5A convertase govern the site of processing in N-cadherin.

### Example 4

### Cleavage of N-Cadherin by Furin or Pc5a Determines the Extent of Cellular Migration

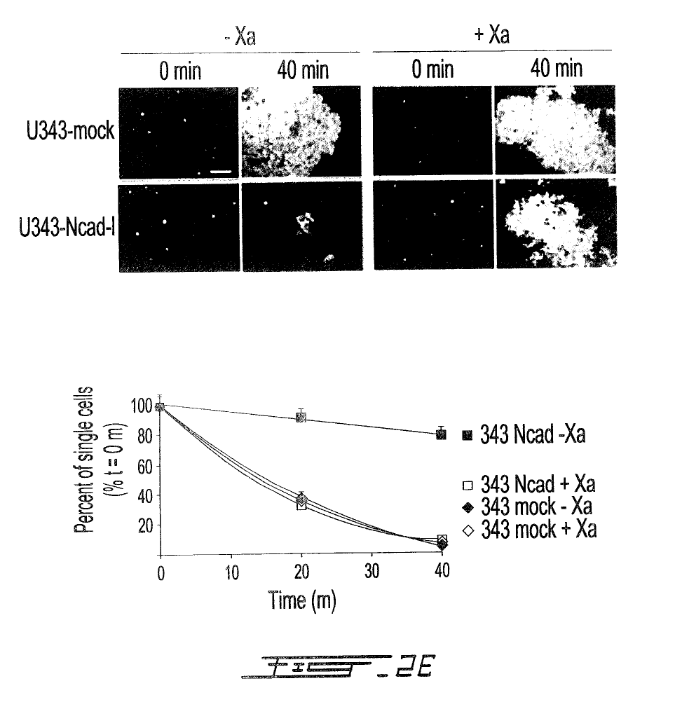

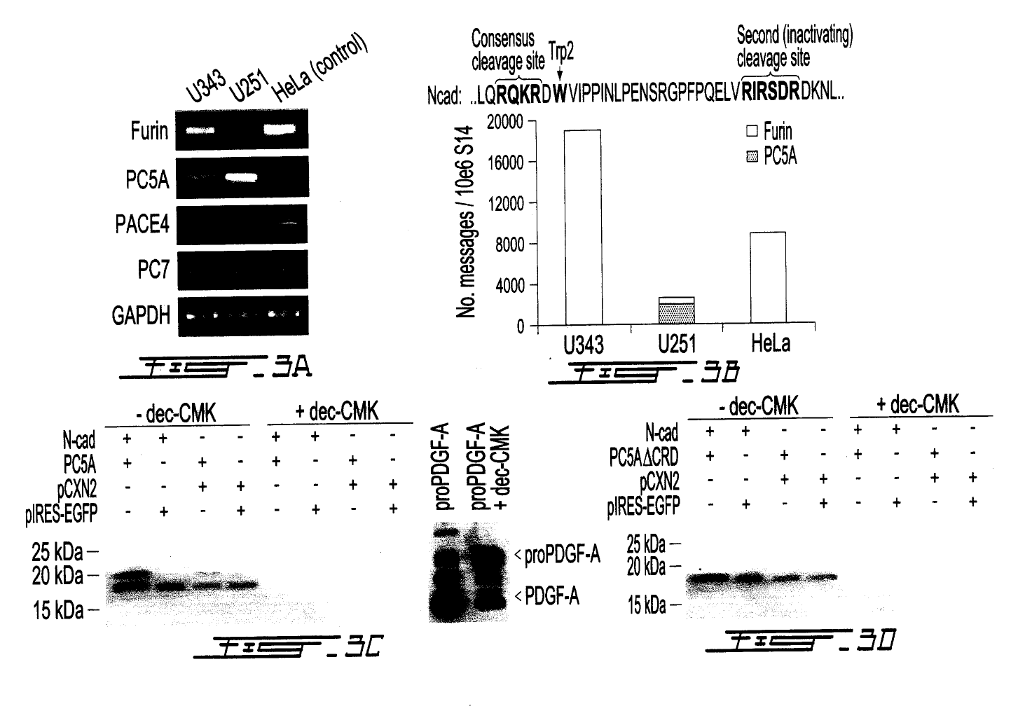

We investigated at which site(s) N-cadherin is processed by endogenous PCs in glioma cells by transfecting these cells with the N-cadherin construct. We were able to detect N-cadherin processing mostly at the second site in U251 cells, and to a much lesser extent at the consensus site (FIG. 4A). This is in contrast to U343 cells, where cleavage appears to take place exclusively at the consensus site (FIG. 4A). As expected, there were no cleavage products detected when the dec-cmk inhibitor was added to the culture medium (FIG. 4A, lanes 4-6). These results are consistent with the PC expression profiles we found for these cell lines (see FIGS. 3A and 3B); however, they appear to be conflicting with the presence of proN on the surface of U251 cells (see FIG. 1E, F). This inconsistency is explained by the fact that due to low furin levels, most of the N-cadherin expressed is brought to the cell surface in the precursor form, and a snapshot of the cell surface would reveal a percentage of precursor N-cadherin (45%, see above), a small percentage of properly cleaved N-cadherin, and the remaining surface N-cadherin would be cleaved at the second site by PC5A. It is also possible that the prodomain fragment resulting from cleavage at the second site remains associated with the cell surface.

Thus relatively high furin levels and low PC5A levels would be expected to render brain tumor cells less invasive and more adhesive to one another since cells would be cleaved at the consensus site, and the contrary would be true for highly invasive brain tumor cells expressing low furin and high PC5A levels.

We looked at the localization of endogenous PC5A by immunocytochemistry, and detected substantial levels of the enzyme at the cell surface of U251 cells, but very low levels at the surface of U343 cells (FIG. 4B). As we observed with HeLa cells, PC5A was not detected at the cell surface when heparin was added to the cells, or when cells were stained under permeabilizing conditions (FIG. 4B). Precursor PC5A (pro-PC5A) was detected with an antibody specific for the prodomain of PC5A (Nour et al. (2005) Molec Biol Cell 16; 5215-5226), and we found that a small fraction of PC5A on the surface of U251 is in the precursor form, and nearly no PC5A was detected on the surface of U343 cells (FIG. 4B).

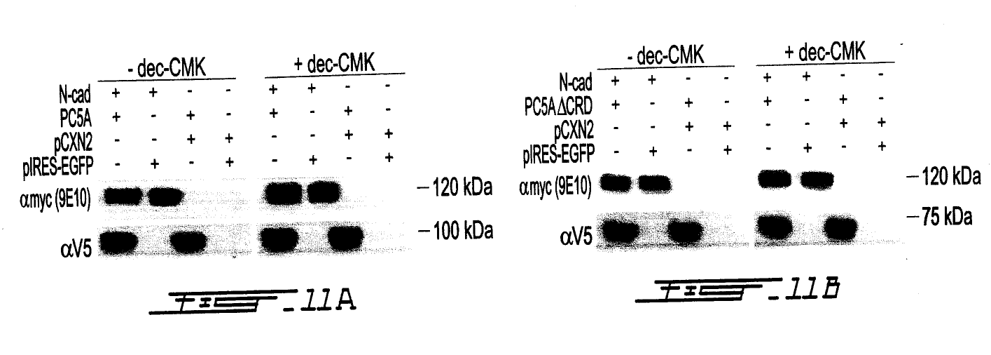

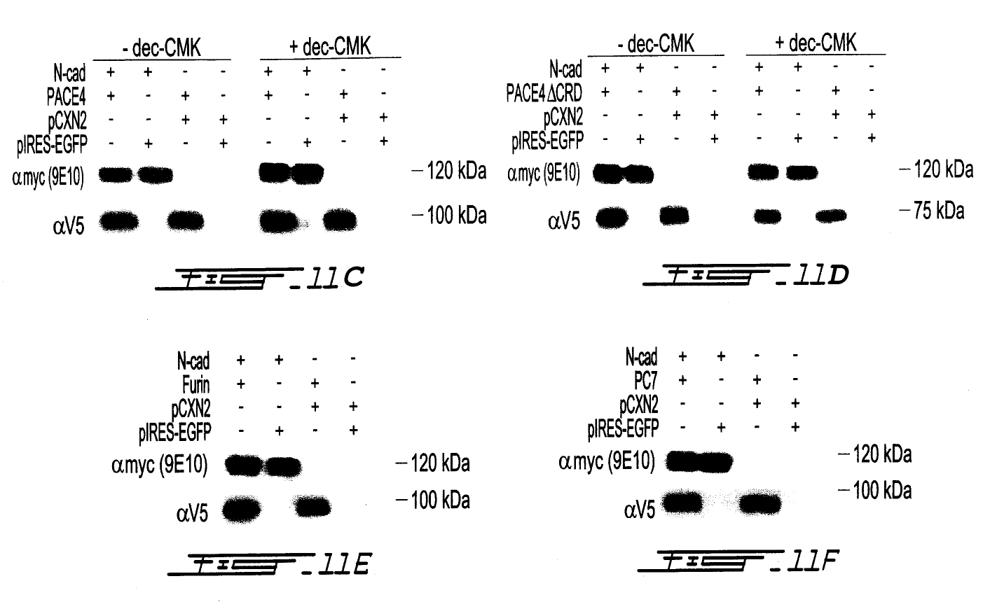

To determine the effects on cellular behavior by furin and PC5A, we carried out gain of function experiments where U251 cells were stably transfected with furin, and U343 cells were stably transfected with PC5A or a catalytically inactive PC5A mutant (PC5A-R84A; (Nour et al. (2003) J Biol Chem 278; 2886-2895). Transfected cells exhibited colocalization of furin or PC5A with EGFP (FIG. 12A). In U343-PC5A cells, the enzyme was detected at the cell surface, mainly in its active form, and as expected, PC5A was in its inactive heterodimeric form (Nour et al. (2005) Molec Biol Cell 16; 5215-5226) on the surface of U343-R84A cells (FIG. 12B). Our results show that compared to mock transfections, overexpression of furin in U251 cells resulted in a substantial decrease in the number of migrated cells at 12 h in a wound healing assay (FIG. 4C). In contrast, overexpression of PC5A in U343 cells resulted in a significant increase in the number of migrated cells compared to mock-transfected cells, and as expected, this effect was not seen with cells transfected with PC5A-R84A (FIG. 4C). U251 cells transfected with furin aggregated to a much greater extent compared to control cells, and U343 cells transfected with PC5A formed very few small aggregates compared to control cells (FIGS. 4D/15C). Thus, the changes observed in migratory behavior are consistent with an effect on cell aggregation.

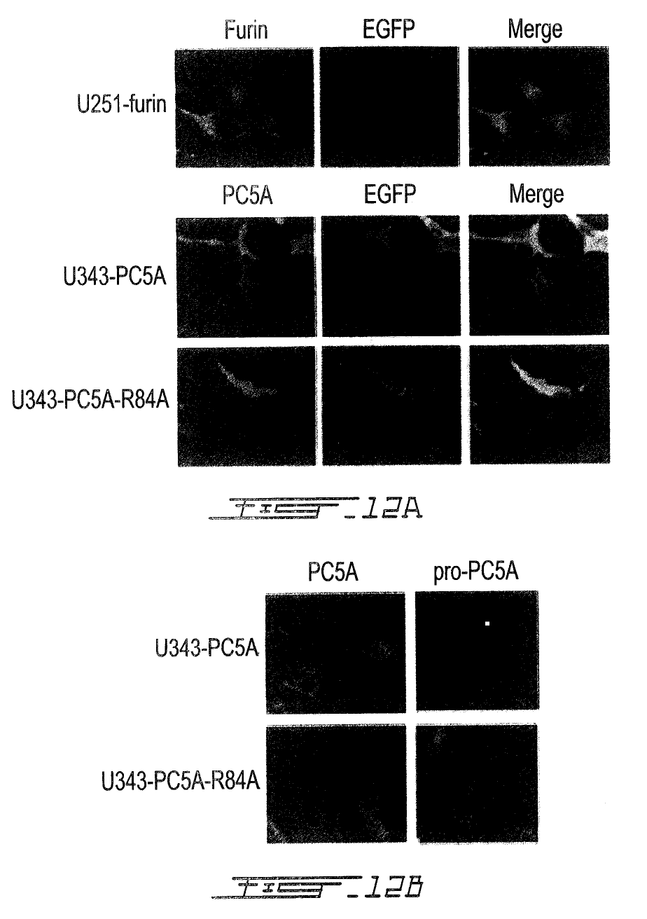

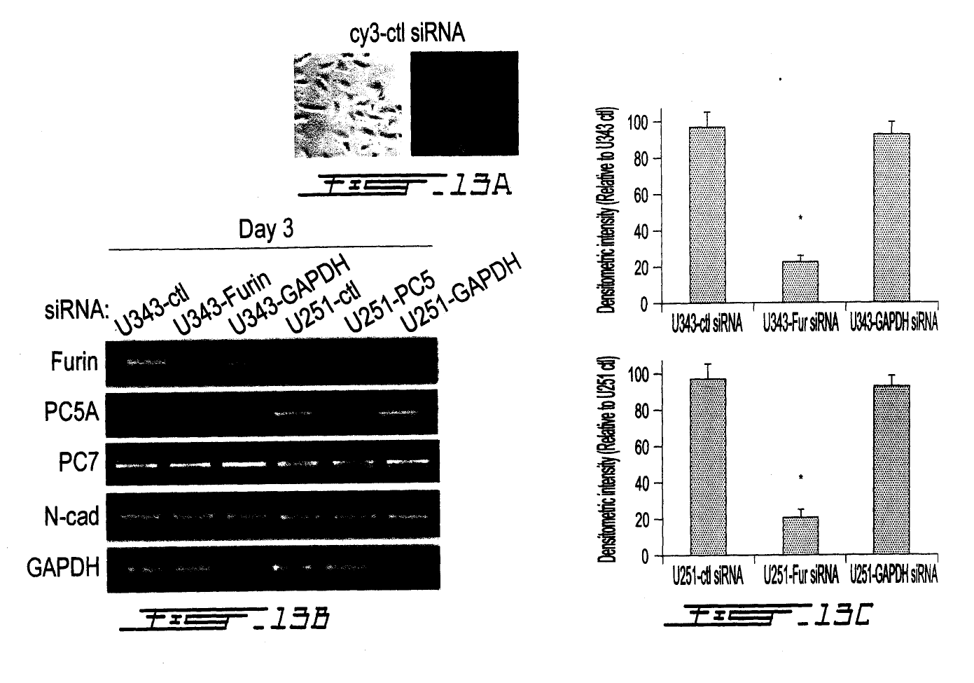

We also carried out knockdown experiments using siRNAs specific for PC5A or furin. Cells were successfully transfected with siRNA (FIG. 13A), and RT-PCR demonstrated an 80% reduction of furin mRNA levels in U343 cells and PC5 mRNA levels in U251 cells (FIGS. 13B and C). Furin or PC5 siRNA did not affect PC7 or N-cadherin mRNA levels (FIG. 13B). In addition, GAPDH levels were not affected by furin or PC5 siRNA, but were reduced by a GAPDH-specific siRNA (FIG. 13B). Immunocytochemistry also demonstrated a reduction in furin and PC5A levels in U343 and U251 cells, respectively (FIG. 13D), but there was no reduction in tubulin, nestin or β-catenin expression (FIG. 13D). Our siRNA results show that compared to ctl siRNA, knockdown of PC5A in U251 cells resulted in a significant decrease in cell migration (FIG. 4C), and a corresponding increase in cell aggregation (FIGS. 4D/15C). The effect on migration was less pronounced than the effect observed with furin-transfected cells (see FIG. 4C). This can be explained by the fact that U251 cells transfected with PC5A siRNA would still express proN on their surface due to low furin expression. Knockdown of furin in U343 cells resulted in a substantial increase in cell migration (FIG. 4C), and decrease in cell aggregation (FIGS. 4D/15C).

Together, these results demonstrate that furin expression appears to inhibit glioma cell migration, and PC5A expression promotes glioma cell migration.

### Example 5

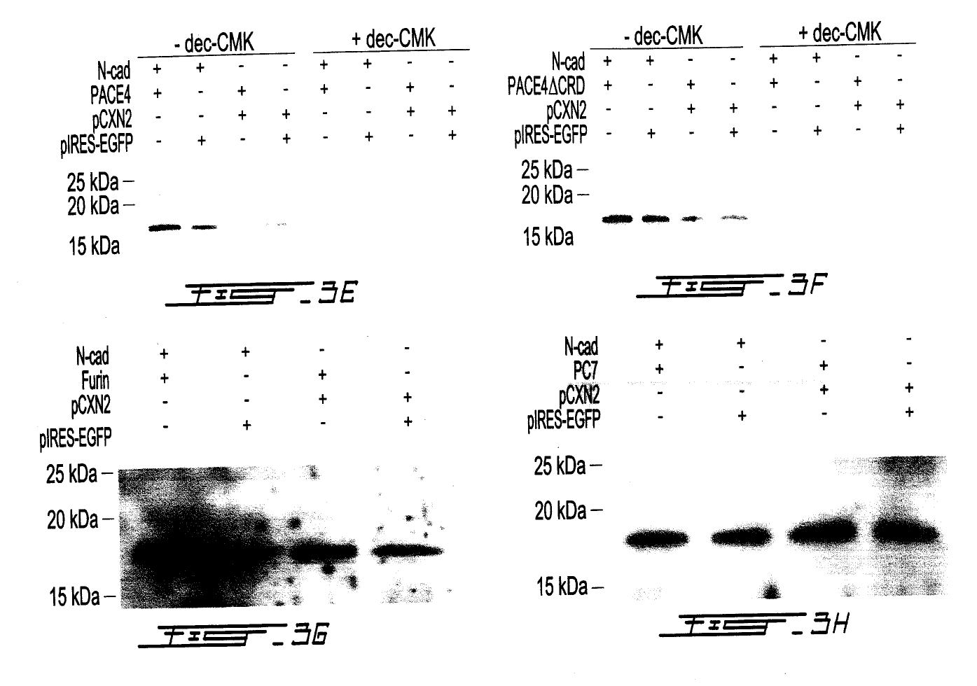

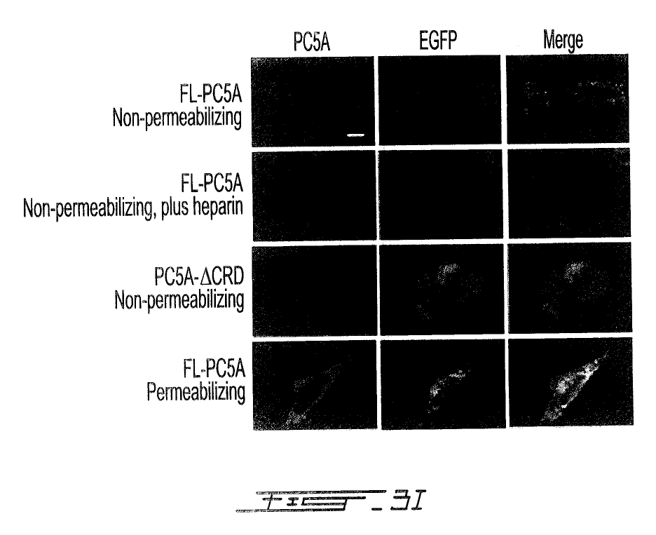

To demonstrate the functional importance of N-cadherin processing by PC5A or furin, we engineered another N-cadherin mutant where the second cleavage site was abolished (Ncad-II), but the consensus site was intact (FIG. 5A). We then carried out a series of transient transfections in U343 cells, which express low levels of PC5A, with either wt N-cadherin, Ncad-I, or Ncad-II alone or in combination with furin or PC5A. Compared to untransfected cells, there is a small decrease in migration (FIG. 5B) and a small increase in aggregation (FIG. 5C) in cells transfected with wt N-cadherin. This was expected since U343 cells express high levels of furin needed to process N-cadherin at the consensus site. A 15% further decrease in migration and increase in aggregation was observed in cells transfected with wt N-cadherin and furin (FIGS. 5B and 5C). There was a large increase in migration and decrease in aggregation when cells were transfected with wt N-cadherin and PC5A, or with proN (as seen in FIG. 2C), or with proN even in the presence of furin (FIGS. 5B and 5C). Importantly, when cells were transfected with Ncad-II and PC5A, the increase in migration detected with wt Ncad and PC5A was not observed (FIG. 5B).

### Example 6

### Correlation of Tumor Aggressiveness and Metastasis with Surface proN and PC Expression in Human Brain Tumor Biopsies and Carcinomas

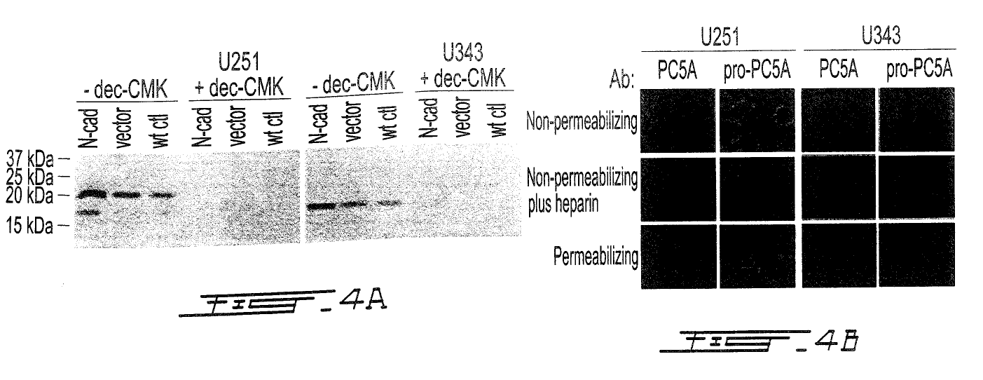

We investigated the surface proN profile as well as PC5A and furin levels in human brain tumor biopsies and several types of carcinoma cell lines (prostate, bladder, squamous cell, and breast) that undergo an E-to-N transition. We found that a high proportion of surface proN was present in highly aggressive glioblastoma multiforme (GBM) cells (˜80%, FIG. 6A). There was a lower proportion of surface proN in a recurrent anaplastic oligodendroglioma (˜50%), and no detectable surface proN in an anaplastic astrocytoma and a low-grade glioma (FIG. 6A). The proportion of surface proN ranged from 40% to 80% in metastatic carcinoma cell lines (FIG. 6B).

We also determined quantitative expression levels of furin and PC5A in the same carcinoma cell lines as well as in a series of human brain tumor biopsies. We found that the metastatic carcinoma cells, including WM266 metastatic melanoma, expressed low furin levels and low PC5A levels (FIG. 6D). In contrast, VGP melanoma cells (WM-115) were found to express relatively high PC5A levels and low furin levels. It was remarkable that all cell lines except those established from metastasized tumor cells, expressed either high furin or PC5A levels, but substantial levels of both enzymes in one cell type were not observed. Clearly the situation in vivo is very complex, since some human brain tumors such as GBMs are very heterogeneous. We found that in general higher grade brain tumors (CT-001, OP-132, OP-122) had lower levels of furin compared to low grade brain tumors (OP-71, CT-005); however, levels of furin were higher than in invasive U251 cells, or in carcinoma cell lines (FIG. 6C). PC5A levels were elevated in higher grade brain tumors, except for OP-71 which had PC5A levels similar to OP-132 (FIG. 6C). Similar to the metastatic carcinoma cell lines, a metastasis to the brain from a malignant breast tumor was found to have relatively low levels of furin and PC5A (FIG. 6C).

These results indicate that it may be possible for N-cadherin to be cleaved by furin intracellulary, and then inactivated by PC5A at the cell surface in cells expressing both enzymes. Sequential cleavage of a precursor protein by PC enzymes has been previously demonstrated. Pro-BMP-4 undergoes serial cleavage at two sites in its prodomain, and differential use of the upstream site determines the activity of the mature protein partially via regulating protein stability (Cui et al. (2006) Genes Develop. 15; 2797-2802). Therefore in a proportion of highly aggressive tumor cells, N-cadherin may be cleaved sequentially by furin and PC5A.

Taken together, our results indicate that cleavage of N-cadherin by furin and PC5A convertases appears to have opposing effects on intercellular adhesion and cellular motility. Furthermore, it appears that PC5A expression is important for cells that are actively invading, and in the process of metastasis, but not for tumor cells that have successfully metastasized to a secondary site. Cleavage of N-cadherin by PC5A has a profound effect on cell motility and is key for invasion. Our results suggest also that a decrease in PC5A expression is an early event necessary for cells to associate with their neighbors and stop invading.

### Example 7

### Certain Common. Human Epithelial Derived Tumors Express Cell Surface proNCAD

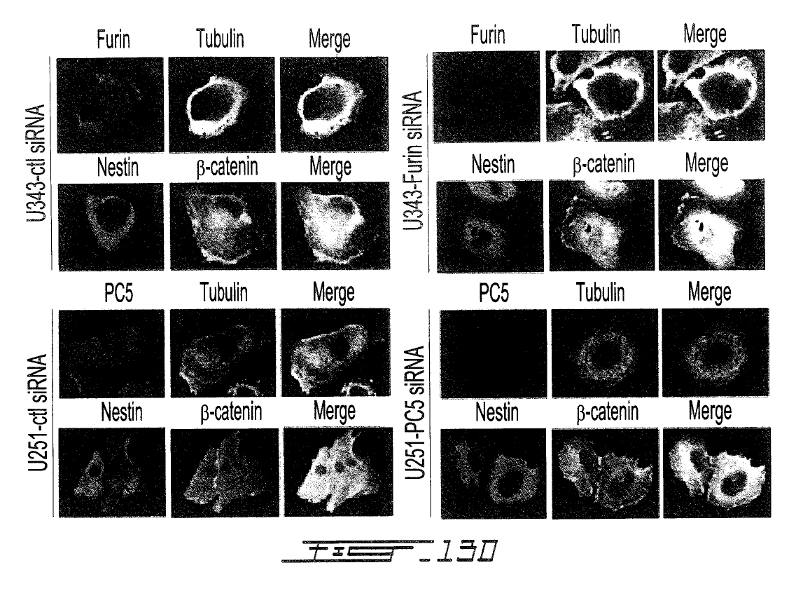

Using a rabbit polyclonal antibody specifically against the NCAD prodomain (anti-proN) (Koch et al., 2004), we examined a panel of carcinoma cell lines derived from post-metastatic sites (prostate (PC3 and PPC-1), bladder (T24 and JCA-1), squamous cell (NC1-H226), and breast (MDA-MB-436)) for surface proNCAD expression. We found that proNCAD could be detected on the surface of these metastatic carcinoma cell lines in cell surface biotinylation experiments and the ratio of surface versus total cell ranged from 40% to 80% (FIG. 14A). We were interested in further pursuing melanoma, a tumor model that undergoes an ECAD to NCAD transition, and glioma, a tumor model that exhibits persistence of NCAD during malignant progression, respectively.

To this end, we made use of melanoma cell lines representing different stages of transformation. WM115 was derived from VGP melanoma at the primary tumor site and WM266 was derived from metastatic melanoma at a secondary site in the same patient. We also used the U343 and U251 glioma cell lines, isolated from a grade III anaplastic astrocytoma, and a GBM, respectively. The U343 and U251 cell lines exhibit different degrees of invasiveness in a collagen gel matrix. U343 cells only invade approximately 500 μm compared to 1400 μm for U251 cells 5 days post-implantation (FIG. 8). We found that although total NCAD levels were comparable in both melanoma cell lines, expression levels of proNCAD were higher in post-metastatic WM266 cells (FIG. 14B). In addition, as assessed by densitometry, we found that a high proportion (˜three fold compared to U343) of proNCAD was present on the surface of highly invasive U251 cells, but not of the indolent U343 cells (FIG. 14C). We looked at immunolocalization of proNCAD and found that it could be detected intracellularly in permeabilized glioma and melanoma cells (FIG. 14D, top panels). Under live cell, non-permeabilizing conditions, we found that proNCAD was detected on the surface of U251 and WM266 cells, and to a lesser extent on the surface of WM115 cells (FIG. 14D, bottom panels), and co-existed with mature NCAD (FIG. 10). ProNCAD was not detected on the surface of U343 cells (FIG. 14D, bottom panels), in agreement with our immunoblot analysis.

Since NCAD is an abundant component of melanoma and glioma cell lines, we wanted to examine the intercellular adhesive activity of these cells. We observed greater aggregation in less invasive U343 glioma cells and in WM115 VGP melanoma cells, compared to highly invasive U251 cells and WM266 metastatic melanoma cells, respectively (FIGS. 14E and 14F; FIG. 9A). This is consistent with higher levels of non-adhesive proNCAD in the U251 and WM266 cell lines. There was no cell aggregation in the absence of calcium for all cell lines (data not shown) revealing that calcium-dependent cadherin mediated adhesion is the only intercellular adhesion mechanism of consequence in these cell lines (Takeichi and Nakagawa, Cadherin-dependent cell-cell adhesion, In Curr Protoc Cell Biol, J.S. Bonifacino, ed. (Kyoto, John Wiley and Sons, Inc), 2001). Aggregation assays were also carried out by mixing either tumor cell lines (labelled with Dil) with L cells overexpressing NCAD or ECAD (LE cells or LN cells, labeled with DiO). We observed mutually exclusive segregation of tumor cells from LE cells, and co-aggregation of tumor cells with LN cells (FIG. 9B). Thus, in these aggregation assays, NCAD is a primary mediator of adhesion in both melanoma and glioma cells.

These results strongly suggest that aberrant cell-surface expression of non-adhesive proNCAD is important for tumor cell migration and invasion.

### Example 8

### Overexpression of proNCAD Promotes Glioma and Melanoma Cell Motility

Since NCAD expression has been shown to correlate with increased motility and proNCAD lacks adhesive function, we hypothesized that loss of adhesion due to aberrant surface expression of proNCAD may serve as a mechanism for enhanced motility in brain tumor cells, even in the presence of mature NCAD. We engineered an NCAD construct (proNCAD) where the endogenous consensus proprotein convertase cleavage site (Koch et al., 2004) was replaced with a serum coagulation Factor Xa recognition site in the linker sequence (FIG. 2A), similar to previously reported constructs. Glioma and melanoma cells transfected with mutant GFP tagged proNCAD or mutant myc proNCAD, respectively, were selected for and clonal populations were expanded. Myc and proNCAD co-localized extensively at the plasma membrane of transfected glioma cells, and GFP and proNCAD showed a similar localization in transfected melanoma cells (FIG. 2B). We investigated the effect of surface proNCAD on intercellular adhesion. We found that cells transfected with mutant proNCAD formed considerably smaller aggregates compared to mock transfected cells (FIG. 2E). Treatment with Factor Xa restored cell-to-cell adhesion, resulting in aggregates comparable to those observed with mock transfected cells (FIG. 2E). Results were quantified as percent of single cells over time, demonstrating low aggregation for transfected cells, and high aggregation for mock transfected cells in the presence or absence of Factor Xa, as well as high aggregation of transfected cells in the presence of Xa (FIG. 2E).

### Example 9

### Glioma and Melanoma Cells Expressing Surface Proncad are More Aggressive In Vivo

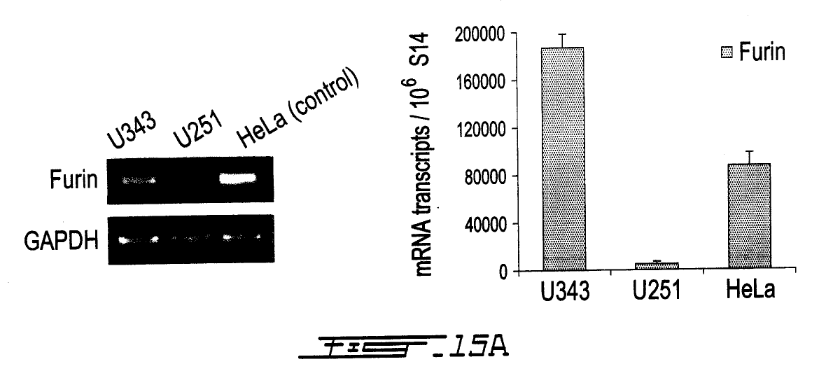

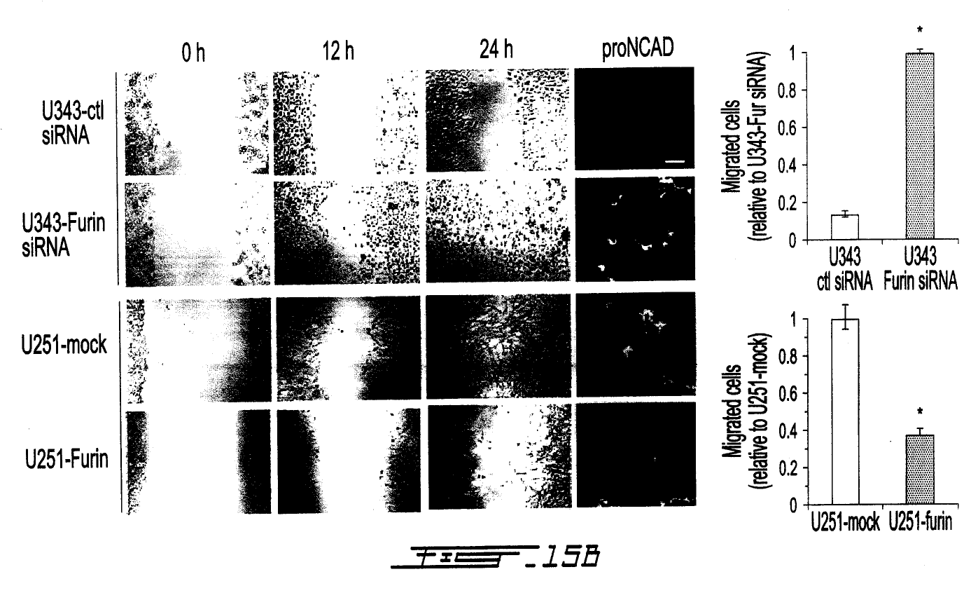

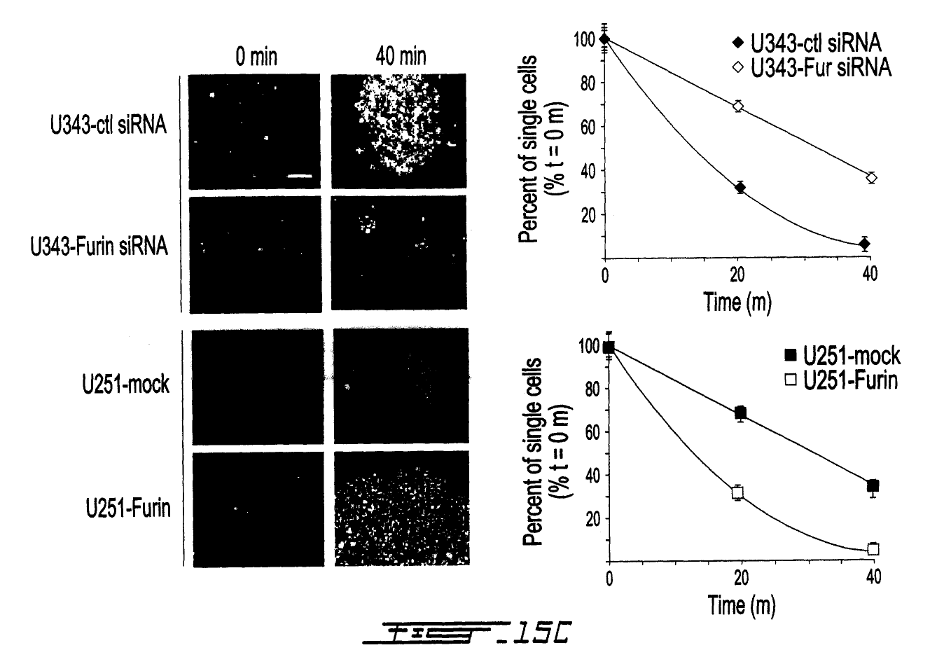

We investigated the effect of surface expressed proNCAD in vivo. U343 glioma and WM266 melanoma cells were transfected with mock vector, wild type (wt) NCAD-myc, or mutant proNCAD-myc, and transfectants were selected. We carried out intracerebral injections of U343 transfectants in the striatum of SCID mice. Tumor growth was analyzed 30 days post-injection using an antibody specifically against human nuclei to detect solid tumor masses and single cells (FIG. 16A). Immunohistochemistry of serial brain sections revealed that tumor cells under all transfection conditions stained positive for human nuclei, Ki67 (MIB-1), and myc (FIG. 16B). As expected, only U343 transfected with the proNCAD mutant exhibited intense proNCAD staining (FIG. 16B). Positive staining for human nuclei representing solid tumor masses and single cells (FIG. 16A) were traced using Neurolucida software and serial sections were used to reconstruct the tumors in three dimensions (FIGS. 17A-17C). U343 cells transfected with mock vector or wt NCAD-myc generally formed a single tumor mass in the striatum of the injected hemisphere (FIGS. 17A and 17B), although tumors formed with wt NCAD-myc cells were slightly smaller due to the strong NCAD-mediated intercellular adhesion. In addition, a relatively small number of single U343 cells transfected with wt NCAD-myc (FIGS. 17B and 17E) were detected in relatively close proximity to the main tumor mass, often migrating along the lateral ventricle. The number of single mock transfected cells detected were not significantly higher (FIG. 17E); however, these cells were also found migrating along the corpus callosum (FIG. 17A). In contrast, U343 cells transfected with mutant proNCAD-myc were much more aggressive, as they formed multiple tumor foci and extensively invaded throughout the brain parenchyma as single cells (FIGS. 17C and 17E). These cells were detected along the ventricles (FIG. 17D, top panel) and the corpus callosum (FIG. 17D, top and middle panels), and invaded the non-injected striatum (FIG. 17D, bottom panel). The mean invasion distance of these cells from the injection site was twice as far compared to the other conditions (FIG. 17F).

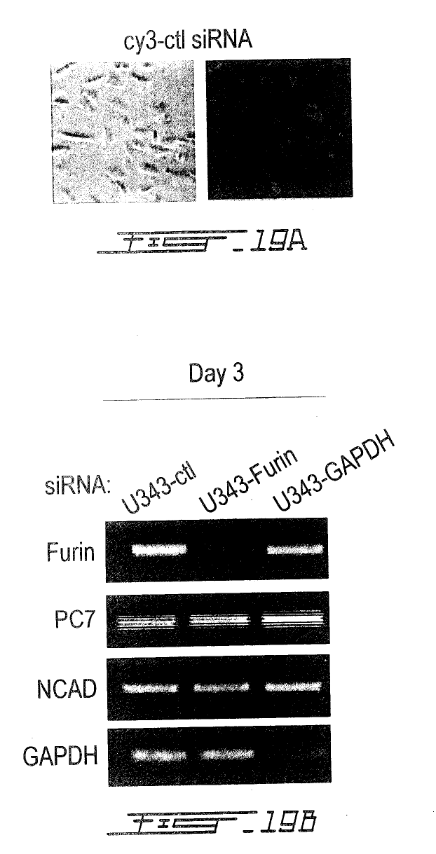

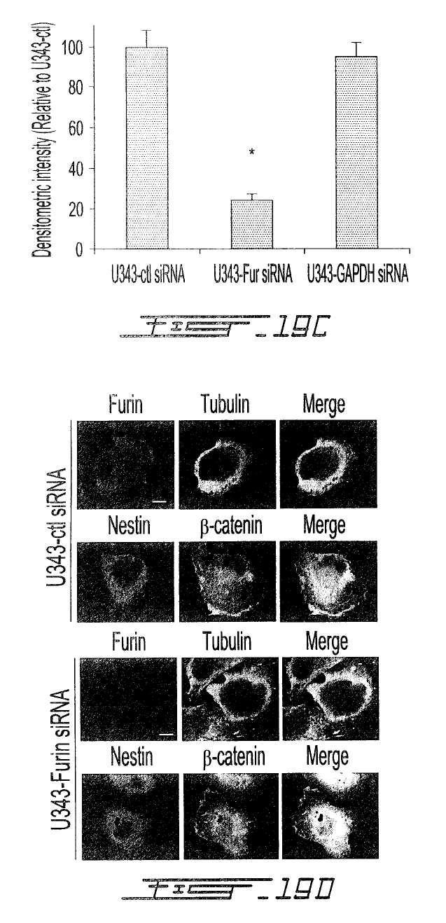

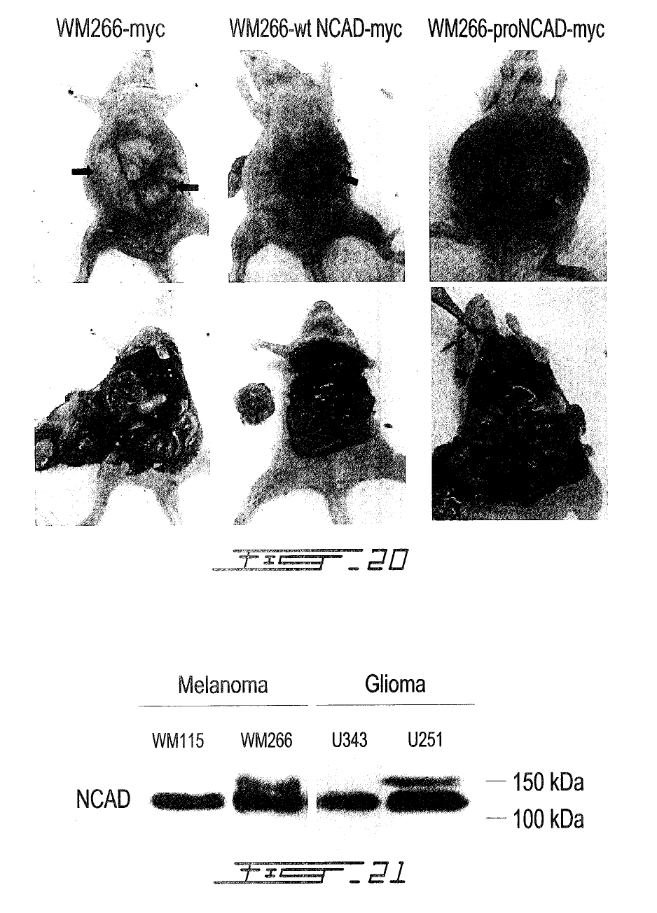

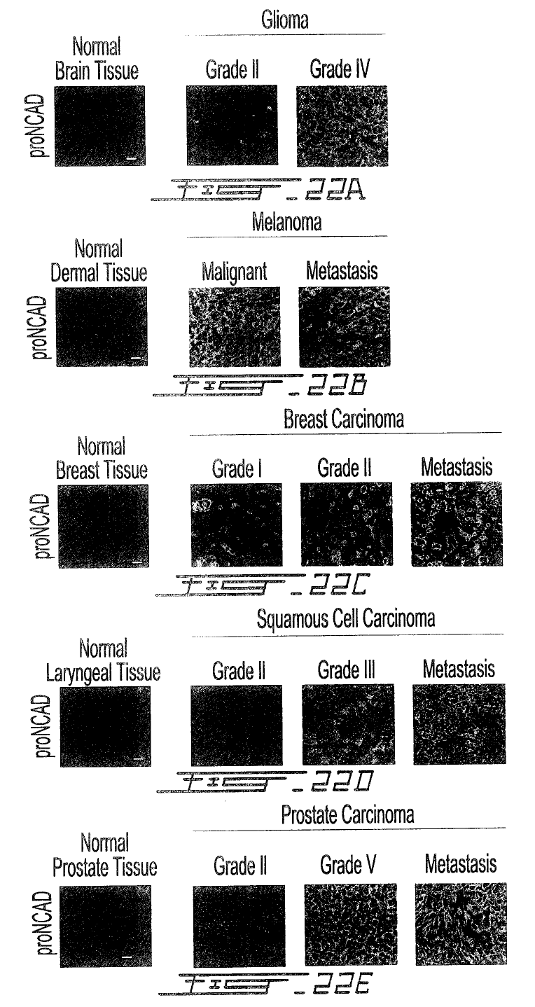

We carried out intra-peritoneal (IP) injections of transfected melanoma cells, and observed tumor growth 30 days post-injection. Pigmented subdermal tumors and several polyps associated with the peritoneum or the small intestine, liver, or spleen, were detected in mice injected with WM266-myc cells (FIG. 20). There were smaller or no subdermal tumors, and fewer or no polyps, in mice injected with WM266 wt NCAD-myc (FIG. 20). Similar to U343 proNCAD-myc, WM266 proNCAD-myc cells were the most tumorigenic, as mice became bloated and developed ascites, and were found to have numerous polyps associated with the peritoneum, liver, spleen, diaphragm, small and large intestine, and stomach (FIG. 20).

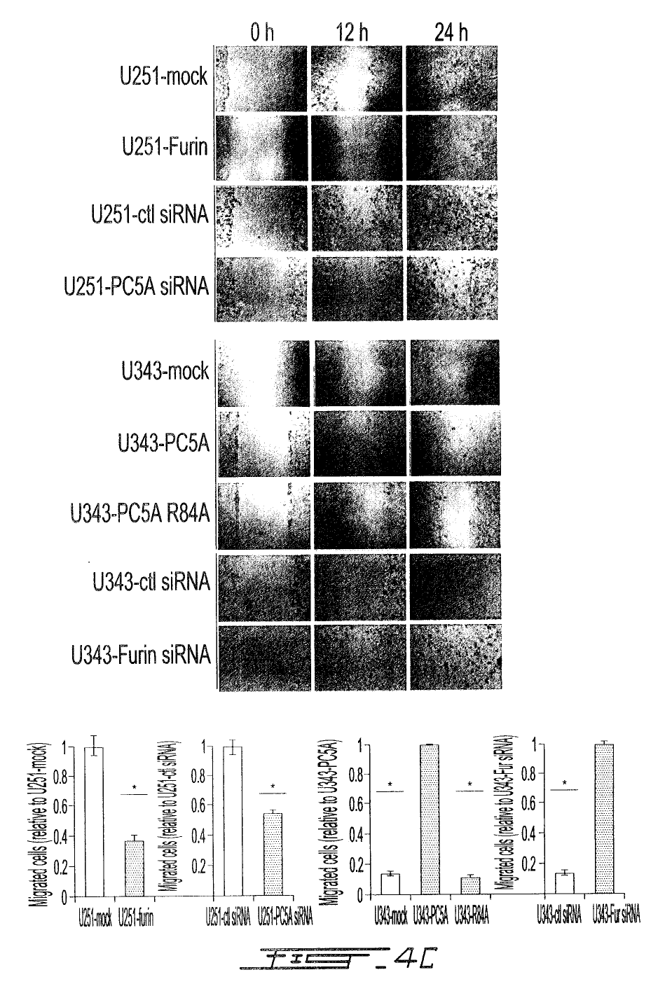

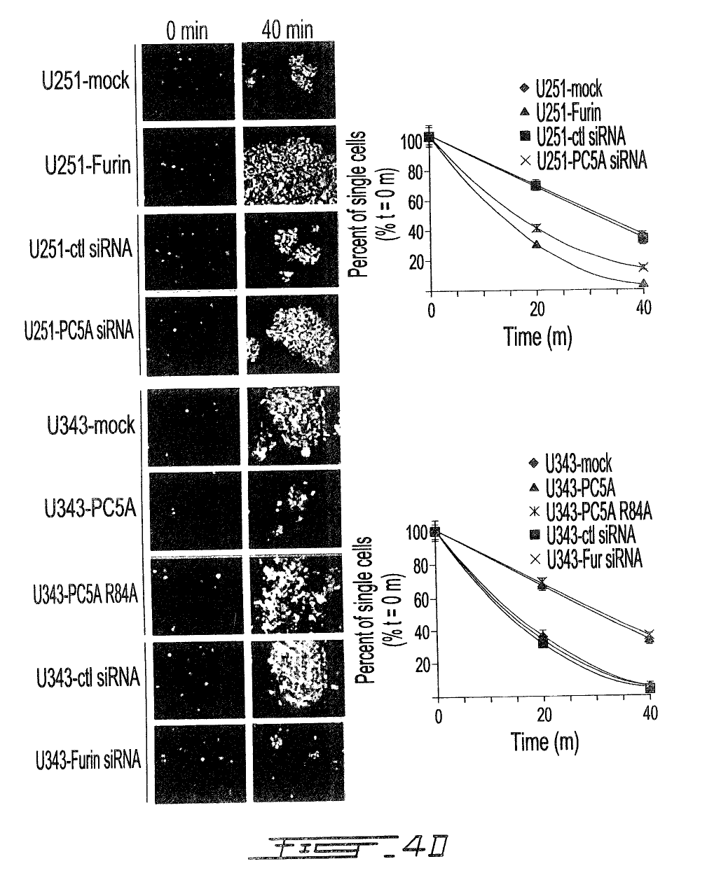

Altogether our results demonstrate that during malignant glioma and melanoma transformation there are significant amounts of non-adhesive proNCAD that appear on the cell surface, in addition to functional NCAD (FIG. 7). We also show that cell surface proNCAD potentiates invasiveness and tumorigenesis in these cells, both in vitro and in vivo.

### EXPERIMENTAL PROCEDURES

### Cell Culture and Transfections

Human WM115, and WM266-4 melanoma cell lines, human U343, and U251 glioma cell lines, and NC1-H226 (squamous cell), and MDA-MB-436 (breast) carcinoma cell lines were purchased from American Type Culture Collection (Rockville, Md.). PPC-1, PC3, JCA-1, and T24 cell lines were the kind gift of Dr. A. Bokhoven.WM115 is a vertical growth phase (VGP) melanoma, and WM266 is a metastatic melanoma. Human U343 and U251 cells, as well as L cells were cultured in DMEM (Gibco) supplemented with 10% FBS (Gibco). Human WM115 and WM266 cells were cultured in MEM (Gibco) supplemented with 2 mM L-glutamine, Earle's BSS, and 10% FBS, and adjusted to contain 1.5 g/L sodium bicarbonate, 0.1 mM non-essential amino acids and 1.0 mM sodium pyruvate. Human MDA-MB-436 cells were cultured in DMEM supplemented with 10% FBS, 10 mcg/ml insulin, and 16 mcg/ml glutathione. Human NC1-H226 cells were cultured in RPMI 1640 medium with 2 mM L-glutamine, 10% FBS, , and adjusted to contain 1.5 g/L sodium bicarbonate, 4.5 g/L glucose, 10 mM HEPES, and 1.0 mM sodium pyruvate. PPC-1, PC3, JCA-1, and T24 human cell lines were cultured in Ham's F12K medium with 2 mM L-glutamine, and 10% FBS, and adjusted to contain 1.5 g/L sodium bicarbonate. All cell lines were cultured in 100 U/ml penicillin, and 100 mg/ml streptomycin, and maintained at 37° C. in a humidified atmosphere of 5% CO2.

N- or E-cadherin-expressing (also referred to herein as NCAD or ECAD expressing, respectively) mouse L cells were generated as previously described (Koch et al. (2004) Structure 12: 793-805). Lipofectamine Plus transfection reagent (Invitrogen) was used to transfect WM115, WM266, and U343 cells with mutant N-cadherin, as well as for transient transfections of HeLa cells and glioma cells. For selection of stable cell lines, cells were seeded in complete DMEM containing 800 μg/ml of Geneticin G418 (GIBCO), the day following transfection.

### Wound-Healing Assays

To assess 2D migration of brain tumor cell lines, 3×105 cells were seeded in chamber slides (Lab Tek), and allowed to grow to confluence. Monolayers were disrupted by scraping with a fine pipette tip, and migration was monitered. Factor Xa was added at a concentration of 0.4 U/ml, where applicable.

### Three-Dimensional Collagen Gel Invasion Assays

Confluent monolayers of tumor cell lines were dissociated and spheroids were prepared using the hanging drop method as previously described (55-58). Spheroids were implanted into 4-well culture dishes containing 0.5 ml aliquots of a collagen type I solution (Vitrogen 100, Cohesion, Palo Alto, Calif.), using a Pasteur pipette. After polymerization at 37° C. for 60 min, the gel was overlaid with 0.5 ml supplemented DMEM. Cell invasion was assessed daily using an inverted phase contrast light microscope. The number of cells invading at increasing distances away from the spheroid was assessed using a concentric grid system (Northern Eclipse 6.0). Factor Xa was added at 0.4 U/ml during spheroid preparation and post-implantation into the collagen gel.

### Boyden Chamber Invasion Assays

To assess cellular invasion, 3×105 cells were seeded on the upper chamber of Matrigel coated membranes (8 μm pore size) (Millipore). Conditioned medium was made by incubating NIH 3T3 cells in DMEM with 0.1% bovine calf serum (BCS) and 50 pg/ml ascorbic acid for 24 h, and was applied to the bottom chamber, serving as a chemoattractant. The cells were allowed to invade the Matrigel substrate for 24 h. The remaining cells that did not migrate through the membrane pores were removed with a cotton swab, and the number of invaded cells was counted in three independent experiments. Factor Xa was added at a concentration of 0.4 U/ml, where applicable.

### Analysis of Proprotein Convertase Expression in Cell Lines and Tissues

RNA was extracted from human cell lines and human brain tumor biopsies using RNeasy mini kit (Qiagen). cDNAs were prepared using the RevertAid™ H Minus First Strand cDNA Synthesis Kit (Fermentas). Human brain tumor tissue was kept frozen at −80° C. until RNA extraction was performed. Semi-quantitative PCR was carried out to determine furin, PC5, PC7, and PACE4 expression, and GAPDH was used as a normalizing control. Real-time PCR was carried out to quantify furin and PC5 expression relative to S14 expression, as previously described (Dubuc et al. (2004) Arterioscler Thromb Vasc Biol 24; 1454-1459). Primers for semi-quantitative and real-time PCR are listed in Table 1.

### Analysis of N-Cadherin Proteolytic Processing

To assess N-cadherin proteolytic processing, we devised an assay where N-terminal cleavage products were detected in the conditioned medium of cells using the proN antibody. Cells were transiently transfected with the appropriate construct(s), the conditioned medium was collected 36 h later, concentrated, and total protein concentration was determined using a Lowry assay (Biorad). Conditioned medium (15 μg protein) was run on a 15% gel, and cleavage products detected were as follows: a 17 kDa band corresponded to cleavage at the consensus site, and a 20 kDa band represented cleavage at the second site.

### Small Interfering RNA

Pre-designed, small interfering RNA (siRNA) for furin (# 105594 and # 112945), PC5A (# 17520 and # 144223), GAPDH, and Cy3-labeled negative control #1 were purchased from Ambion. U343 and U251 cells were transfected with furin siRNA (80 nM) and PC5 siRNA (80 nM), respectively, using Lipofectamine plus reagent. Cells were used in experiments 3 days after transfection.

### Statistical Analysis

Descriptive statistics including mean, standard error of the mean, analysis of variance (ANOVA), independent sample t-tests and Tukey's test for multiple comparisons, were used to determine significant differences between pairs. P values less than 0.05 were considered significant.

### Antibodies and Reagents

The following primary antibodies were used for Western blots and immunocytochemistry: rabbit affinity purified polyclonal anti-N-cadherin cytoplasmic domain, and anti-N-cadherin pro-region (Koch et al. (2004) Structure 12: 793-805); generated in Dr. D. R. Colman's laboratory), rat monoclonal anti-N-cadherin extracellular domain (NEC2) (Dr. Takeichi, RIKEN, Japan), mouse monoclonal anti -GFP (Clontech/BD), rabbit polyclonal anti-PC 5A, anti-propC5A, anti-furin, and anti-PC7 (Dr. N.G. Seidah), mouse monoclonal anti-myc (9E10; Sigma), mouse monoclonal anti-erk (Upstate Biotechnology), mouse monoclonal anti-V5 (Invitrogen), mouse monoclonal anti-tubulin (Upstate), rabbit polyclonal anti-nestin (Chemicon), and mouse monoclonal anti-β-catenin (Upstate). Fluorescent-conjugated secondary antibodies were from Chemicon. DAKO (cytomation fluorescence mounting media; Dakocytomation) was used to mount coverslips on glass slides. Lipophilic dye Dil (1,1-dioctadecyl-3,3,3,3,-tetramethylindocarbocyanine), and DiO (3,3-dioctadecyloxacarbocyanine perchlorate) were purchased from Molecular Probes. Decanoyl-Arg-Val-Lys-Arg-chloromethylketone (Dec-cmk) was purchased from Bachem.

The following primary antibodies were used for Western blots, immunocytochemistry, and immunohistochemistry: rabbit affinity purified polyclonal anti-NCAD cytoplasmic domain, and anti-NCAD pro-region ((Koch et al., 2004); generated in D. R. C. laboratory), rat monoclonal anti-NCAD extracellular domain (NEC2) (Dr. M. Takeichi, RIKEN, Japan), mouse monoclonal anti -GFP (Clontech/BD), mouse monoclonal anti-myc (9E10; Sigma), mouse monoclonal anti-ERK (Upstate Biotechnology), rabbit polyclonal anti-furin (N. G. S. laboratory), mouse monoclonal anti-tubulin (Upstate), rabbit polyclonal anti-nestin (Chemicon), mouse monoclonal anti-p-catenin (Upstate), mouse monoclonal anti-human nuclei (Chemicon), and rabbit polyclonal anti-Ki 67 (MIB-1) (Abcam). Fluorescent-conjugated secondary antibodies were from Chemicon. Fluorescence mounting media (DAKO) was used to mount coverslips on glass slides. Primary antibody enhancer, HRP polymer secondary solution (anti-mouse and anti-rabbit), and the AEC chromogen were from Lab Vision. Aquatex mounting media was from EMD Chemicals. Lipophilic dye Dil (1,1-dioctadecyl-3,3,3,3,-tetramethylindocarbocyanine), and DiO (3,3-dioctadecyloxacarbocyanine perchlorate) were purchased from Molecular Probes.

### Constructs

A wild type N-cadherin cDNA, and a mutant N-cadherin myc- or GFP-tagged cDNA (Ncad-1) was as previously described (Koch et al. (2004) Structure 12: 793-805). An N-cadherin myc-tagged cDNA mutated at the second cleavage site (Ncad-II) was generated using QuickChange II XL site-directed mutagenesis kit (Stratagene), according to manufacturer instructions. Mutagenesis primers were as follows: 5′GTCAGAATCAGGTCTGATGCAGATAAAAACCTTTCCC 3′ (forward), and 5′ GGGAAAGGTTTTTATCTGCATCAGACCTGATTCTGAC 3′ (reverse). Wild type PC5A, and PACE4 EGFP— and V5-tagged cDNA, as well as cDNAs of PC5A and PACE4 with the CRD deleted, were as previously described (Nour et al. (2005) Mol. Biol. Cell 16: 5215-26). V5-tagged furin cDNA and PC7 cDNA were cloned into the pIRES2-EGFP vector (Seidah et al. (1999) Ann N Y Acad Sci 885; 57-74).

### Immunoblotting

For protein extraction, subconfluent monolayers were washed with PBS, dissociated using 2 mM EDTA in PBS (as above), and pelleted at 1000 rpm for 5 min. Lysates were obtained using RIPA lysis buffer (150 mM NaCl, 20 mM Tris-HCl, pH 7.5, 1% NP-40, 1% Triton X-100) with protease inhibitors (complete mini, Roche Diagnostics) on ice for 30 min. After cell lysis, samples were centrifuged for 15 min at 15,000 rpm and the supernatants were transferred to clean tubes. Protein concentration was determined using the Lowry assay (Biorad DC protein assay) and samples were run on a 4-15% linear gradient SDS-PAGE gel (Biorad), transferred to nitrocellulose, membrane-blocked with 5% milk protein, and incubated overnight with primary antibodies at 4° C. Blots were then incubated with HRP-conjugated secondary antibodies, and routine washes were carried out. Blots were developed with the chemiluminescence system (Pierce Biotechnology). Alternatively, for signal quantification, the chemifluorescence kit was employed (Pierce Biotechnology) and the Storm Imager.

### Cell-Surface Biotinylation

Subconfluent monolayers were washed three times with ice cold PBS containing 2 mM MgCl2, and incubated with 0.2 mg/ml EZ-Link NHS-SS-Biotin (Pierce Biotechnology) solution in PBS for 30 min at 4° C. to inhibit endocytosis. Excess biotin was quenched by washing three times with ice cold TBS (25 mM Tris-HCl, pH 8.0, 150 mM NaCl, 2 mM MgCl2, and 2 mM CaCl2), followed by 3 washes with ice cold PBS. Cells were scraped off the plate with 0.5 ml RIPA buffer and lysis was carried out as above, followed by protein concentration determination of lysate supernatants. Immunopure Immobilized Streptavidin beads (Pierce) were added to 30 or 60 μg of total protein, the volume brought up to 0.5 ml with RIPA buffer. Binding of biotinylated proteins to streptavidin beads occurred during a 2h incubation at 4° C., with gentle rocking. Streptavidin beads were pelleted (13 000 rpm, 4° C.), the supernatant was discarded and beads were washed with 1 ml RIPA buffer three times. The supernatant from the last wash was discarded and 2×SDS sample buffer containing 100 mM DTT was added to dissociate the biotinylated proteins from the streptavidin beads via reduction of the disulfide bond in the biotin molecule. Samples were run on SDS-PAGE gels and immunoblotting was carried out as outlined above. Anti-N-cadherin cytoplasmic antibody was used to detect total N-cadherin protein (mature and precursor), anti-proN antibody was used to detect precursor N-cadherin, and anti-erk was used as a cell surface biotinylation control.

### Immunocytochemistry

Cells were plated onto poly-L-lysine coated coverslips in supplemented DMEM (see above). Cells were fixed in 4% paraformaldehyde, permeabilized in 0.3% TritonX, PBS, and blocked in 5% BSA, 5% goat serum, PBS. Cells were then incubated for 1 h in primary antibody diluted in 1% BSA, 0.02% TritonX, PBS, followed by a 40 min incubation in fluorescent-conjugated secondary antibodies. Three washes with PBS were performed before fixation, as well as following each step. Coverslips were mounted and examined by confocal laser microscopy using the Zeiss LSM 510 microscope and 60×oil immersion objective.

Live-cell staining was carried out by incubating cells plated on coverslips with primary antibody diluted in medium without serum at 4° C. for 1 h. The cells were washed with PBS and fixed in 4% paraformaldehyde. Following washes with PBS, cells were incubated with fluorescent-conjugated secondary antibody diluted in 1% BSA, 0.02% TritonX, PBS, for 40 min at room temperature. Coverslips were then mounted and examined as above. For surface PC5A staining, cells were washed twice with ice-cold PBS, fixed with freshly prepared 3.7% paraformaldehyde for 10 min on ice, washed 3 times with PBS, incubated in 150 mM glycine for 5 min, washed once with PBS, blocked for 30 min in 1% BSA, incubated in primary antibody overnight at 4° C., washed 4 times with PBS, incubated with secondary antibody for 40 min at room temperature, and washed 4 times with PBS. Coverslips were mounted and examined as above.

### Immunohistochemistry

Briefly, sections were air dried for 30 min to 2h, washed with PBS for 5 min, blocked in PBS containing 10% FBS and 0.5% triton-X-100 for 90 min, and incubated with primary antibody in blocking solution overnight at 4° C. in a humidified chamber. Sections were then washed three times in PBS, incubated in secondary antibody in blocking solution for 90 min at room temperature in a humidified chamber, and washed two times in PBS. Slides were mounted and examined by confocal laser microscopy using the Zeiss LSM 510 microscope. Alternatively, sections were incubated in 0.1% Triton X-100 for 10 min, in 0.3% v/v hydrogen peroxide, and blocked in 1% goat serum in PBS for 30 min. Blocked slides were rinsed in PBS and incubated with primary antibodies overnight at 4° C. The slides were incubated in the HRP polymer solution, and developed with the AEC chromogen solution according to the manufacturer's recommendations. Sections were counterstained with hematoxylin and coverslipped .

### Cell Aggregation Assays

Monolayer cultures were treated with 2 mM EDTA in PBS for 5 min at 37° C. The cells were then washed gently in HCMF (Hepes-buffered Ca2+-Mg2+-free Hanks' Solution) supplemented with 1 mM CaCl2 and 1% BSA for 30 min at 37° C., to dissociate the monolayer into single cells while leaving cadherins intact on the cell surface. Following cell dissociation, 5×105 cells per well were transferred to 24-well low-adherent dishes (VWR), and brought up to a final volume of 0.5 ml HCMF containing 1% BSA with or without 1 mM Ca2+. The plates were rotated at 80 rpm at 37° C. for 40 min. At t=0 min, t=20 min, and t=40 min, 50 μl of the fixed aggregates were removed, placed on a slide, covered with a coverslip, and examined by light microscopy. For mixed aggregation analysis, tumor cells were labeled with dye Dil, and L cells either expressing N-cadherin or E-cadherin were labeled with DiO. Stock solutions of Dil were made by dissolving 2.5 mg Dil in 1 ml of 100% ethanol, and stocks of DiO were made by dissolving 2.5 mg DiO in 1 ml of 90% ethanol, 10% dimethylsulfoxide. The stock solutions were sonicated and filtered before use. Cell monolayers were labeled with these dyes by incubating them overnight in serum-containing DMEM with either 15 μg/ml Dil or 10 μg/ml DiO. Cells were washed extensively with PBS containing calcium, single cell suspensions were obtained as described above, and 5×105 cells per well of each of two types were transferred to a 24-well dish. The dishes were rotated and aggregates were examined by fluorescent microscopy. Where applicable, Factor Xa (0.4 U/ml, Sigma) was added before and after cell dissociation.

Primary Tumour Cell Cultures from Human Patient Resections

Primary cell cultures were prepared from human brain tumour resections carried out at the Montreal Neurological Hospital (Quebec, Canada) by Dr. Rolando F. Del Maestro. All patients signed a written consent form prior to the operation. Pathology reports classified tumors from patients OP-128 and OP-132 as GBMs, OP-122 as an anaplastic astrocytoma, OP-133 as a recurrent anaplastic oligodendroglioma, and OP-109 as a low grade glioma. Single cell suspensions from these tumour resections were obtained by serial trypsinization. Briefly, the tissue was mechanically dissociated using a scalpel, in a Petri dish containing PBS, placed in a conical tube with 0.25% trypsin, and DMEM (1:1), shaken, and placed in a 37° C. water bath for 5 min, allowing the tissue to settle to the bottom of the tube. The supernatant, containing suspended tumour cells, was transferred to a clean tube, pelleted, resuspended in supplemented DMEM with 20% FBS, and plated. Two more rounds of trypsinization were carried out on the remaining tissue pieces, and each time the pelleted cells were plated.

The tissue biopsy samples were kept at −80° C. in the Brain Tumor Tissue Bank, in the Brain Tumor Research Centre (BTRC), Montreal Neurological Institute (MNI), and used for RNA isolation and determination of furin and PC5 expression: CT-01-001 and OP-132 were GBM, OP-122 was an anaplastic astrocytoma (III), OP-71 was a low grade glioma, CT-04-005 was a ganglioglioma, and OP-113 was a metastatic breast carcinoma.

### In Vivo Tumor Cell Injections

Intra-peritoneal injections were completed using female, 6 week old, CD1 nu/nu athymic mice (Charles River Canada). 1×106 melanoma cells were suspended in 500 μl of phosphate buffered saline (PBS) and injected into the left lower quadrant of the abdomen.

Stereotactic intra-cerebral tumor cell injections were completed as described (Martuza et al. (1991) Science 252: 854-856). Briefly, female, 6 week old, CD1 nu/nu athymic mice (Charles River Canada) were anaesthetized by intra-peritoneal injection using a cocktail containing ketamine, xylazine and acepromazine. The animal was placed in a stereotactic frame (Kopf Instruments) and a small incision at the midline of the skull was made. A burr hole was drilled 0.5 mm anterior and 2 mm lateral to bregma. A microliter syringe (Hamilton Company) was slowly lowered through the burr hole to a depth of 4.4 mm and a cell suspension consisting of 1×105 cells, as counted by a hemocytometer, in 3 μl of PBS was injected over a 12 minute period. The syringe was slowly withdrawn and the animals were given saline subcutaneously to aid in recovery. Animals were euthanized at one month and tumor invasion was analyzed as described below. All animal experimentation was approved by the Institutional Animal Care Committee and conformed to the guidelines of the Canadian Council of Animal Care.

### Analysis of Intracerebral Injections

Animals were anaesthetized with 2.5% Avertin and perfused intraventricularly using a 4% paraformaldehyde (Pfa) solution. the brain was removed and placed in 4% PFA solution for 15 minutes before being transferred to a 30% sucrose solution overnight at 4° C. The tissue was then embedded into optimal cutting temperature (OCT) and left to freeze overnight at −80° C. 20 μm serial sections of these frozen blocks were taken using a cryostat (Leica Microsystems) and prepared for immunohistochemistry.

All morphometric analysis including 3D reconstruction, invasion distance calculations and tumor foci counts were completed using Neurolucida (MBF Bioscience).

### Statistical Analysis

Descriptive statistics including mean, standard error of the mean, analysis of variance (ANOVA), independent sample t-tests and Tukey's test for multiple comparisons, were used to determine significant differences between pairs. P values less than 0.05 were considered significant.

TABLE 1
Primers used for PCR experiments

| Target | Sequence ( 5′ to 3′) |
|---|---|
| hFurin | ¹(+)-ATCCCAGGAATGAGTTGTC |
| | ²(−)-CTCACCCTGTCCTATAATCG |
| hPC5A | (+)-tgaccactcttcagagaatggatac |
| | (−)-gagatacccactagggcagc |
| hPC7 | (+)-CATCATTGTCTTCACAGCCACC |
| | (−)-ATGACTCATCCCCGACATCC |
| hPACE4 | (+)-GGTGGACGCAGAAGCTCTCGTTG |
| | (−)-AGGCTCCATTCTTTCAACTTCC |
| hGAPDH | (+)-CGAGATCCCTCCAAAATCAA |
| | (−)-CATGAGTCCTTCCACGATACCAA |
| hS14 | (+)-CAGGTCCAGGGGTCTTGGTCC |
| | (−)-GGCAGACCGAGATGAATCCTCA |
| hNCAD | (+)-AGAGGGATCAAAGCCTGGGACGTAT |
| | (−)-TCCACCCTGTTCTCAGGGACTTCTC |

¹(+), forward primer;²(−), reverse primer

The contents of all documents and references cited herein are hereby incorporated by reference in their entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for diagnosing or determining prognosis of a cancer in a subject, comprising determining the molecular form of cadherin at the cell surface of cancer cells in the subject, wherein the presence of a non-adhesive form of cadherin or a high ratio of non-adhesive to adhesive forms of cadherin indicates that the cancer is invasive or metastatic.

2. (canceled)

3. (canceled)

4. A method for monitoring the progression of a cancer in a subject, the method comprising determining the molecular form of cadherin at the cell surface of cancer cells in the subject, wherein the presence of a non-adhesive form of cadherin or a high ratio of non-adhesive to adhesive forms of cadherin indicates that the cancer has progressed to a metastatic phase.

5. A method for monitoring the efficacy of an anti-cancer treatment in a subject, comprising:
determining the molecular form of cadherin at the cell surface of cancer cells in the subject at a first timepoint;
determining the molecular form of cadherin at the cell surface of cancer cells in the subject at a second timepoint; and
comparing the amounts of non-adhesive and adhesive cadherin at the first and second timepoints;
wherein a decrease or no change in the amount of non-adhesive cadherin or an increase in the amount of adhesive cadherin in the second sample compared to the first sample indicates efficacy of the anti-cancer treatment.

6. (canceled)

7. The method of claim 1, wherein said cancer is selected from the group consisting of melanoma, breast cancer, prostate cancer, bladder cancer, squamous cell cancer, and malignant glioma.

8. The method according to claim 1, wherein said cadherin is a type I or type II classical cadherin.

9. The method according to claim 8, wherein said cadherin is selected from the group consisting of E-cadherin, N-cadherin, R-cadherin; C-cadherin, VE-cadherin, P-cadherin, K-cadherin, T1-cadherin, T2-cadherin, OB-cadherin, Br-cadherin, M-cadherin, cadherin-12, cadherin-14, cadherin-7, F-cadherin, cadherin-8, cadherin-19, EP-cadherin (Xl), BS-cadherin (Bs) and PB-cadherin (Rn).

10. The method according to claim 8, wherein said cadherin is N-cadherin.

11. The method according to claim 1, wherein the molecular form of cadherin at the cell surface of cancer cells in the subject is determined using immunocytochemistry or immunoblotting in a sample from a subject.

12. The method according to claim 1, wherein the molecular form of cadherin at the cell surface of cancer cells in the subject is determined using radionuclide imaging, SPECT imaging, magnetic resonance imaging, fluorescence imaging, positron emission tomography, CT imaging, or a combination thereof.

13. A kit for diagnosing or determining prognosis of a cancer in a subject, comprising reagents for determining the molecular form of cadherin at the cell surface of cancer cells in the subject, and instructions for use thereof.

14. The kit of claim 13, wherein the reagents comprise an antibody specific for a non-adhesive cleavage form.

15. The kit of claim 14, wherein the antibody is specific for the pro-domain of a cadherin.

16. The kit of claim 15, wherein the antibody is specific for the pro-domain of N-cadherin.

17. The kit of claim 16, wherein the antibody is anti-proN.

18. The kit of claim 13, further comprising reagents for determining expression levels of furin or PC5 in cancer cells in the subject, and instructions for use thereof.

19. The kit of claim 18, wherein the reagents are PCR reagents, primers, antibodies specific for furin or PC5, and/or reagents for assaying furin or PC5 enzymatic activity.

20. (canceled)

21. (canceled)

22. (canceled)

23. (canceled)

24. (canceled)

25. (canceled)

26. (canceled)


SEQUENCE LISTING (16 sequences)

SEQ ID NO: 1 - DNA, 37 bp, artificial sequence
  primer
gtcagaatcaggtctgatgcagataaaaacctttccc

SEQ ID NO: 2 - DNA, 37 bp, artificial sequence
  primer
gggaaaggtttttatctgcatcagacctgattctgac

SEQ ID NO: 3 - DNA, 19 bp, artificial sequence
  primer
atcccaggaatgagttgtc

SEQ ID NO: 4 - DNA, 20 bp, artificial sequence
  primer
ctcaccctgtcctataatcg

SEQ ID NO: 5 - DNA, 25 bp, artificial sequence
  primer
tgaccactcttcagagaatggatac

SEQ ID NO: 6 - DNA, 20 bp, artificial sequence
  primer
gagatacccactagggcagc

SEQ ID NO: 7 - DNA, 22 bp, artificial sequence
  primer
catcattgtcttcacagccacc

SEQ ID NO: 8 - DNA, 20 bp, artificial sequence
  primer
atgactcatccccgacatcc

SEQ ID NO: 9 - DNA, 23 bp, artificial sequence
  primer
ggtggacgcagaagctctcgttg

SEQ ID NO: 10 - DNA, 22 bp, artificial sequence
  primer
aggctccattctttcaacttcc

SEQ ID NO: 11 - DNA, 20 bp, artificial sequence
  primer
cgagatccctccaaaatcaa

SEQ ID NO: 12 - DNA, 23 bp, artificial sequence
  primer
catgagtccttccacgataccaa

SEQ ID NO: 13 - DNA, 21 bp, artificial sequence
  primer
caggtccaggggtcttggtcc

SEQ ID NO: 14 - DNA, 22 bp, artificial sequence
  primer
ggcagaccgagatgaatcctca

SEQ ID NO: 15 - DNA, 25 bp, artificial sequence
  primer
agagggatcaaagcctgggacgtat

SEQ ID NO: 16 - DNA, 25 bp, artificial sequence
  primer
tccaccctgttctcagggacttctc